(19) United States
(12) Patent Application Publication
(10) Pub. No.: US 2015/0185224 A1
(43) Pub. Date: Jul. 2, 2015

(54) PREDICTING BLADDER CANCER RESPONSIVENESS TO BCG

(71) Applicant: MEMORIAL SLOAN-KETTERING CANCER CENTER, New York, NY (US)

(72) Inventors: Michael Glickman, Pelham, NY (US); Xuejun Jiang, Cresskill, NJ (US); Daniel Barkan, Tel Aviv (IL); Gil Redelman-Sidi, New York, NY (US); Gopa Iyer, New York, NY (US); David Solit, New York, NY (US); Bernard H. Bochner, Scarsdale, NY (US)

(73) Assignee: MEMORIAL SLOAN-KETTERING CANCER CENTER, New York, NY (US)

(21) Appl. No.: 14/420,837

(22) PCT Filed: Aug. 7, 2013
(86) PCT No.: PCT/US2013/053957

§ 371 Date: Feb. 10, 2015

Related U.S. Application Data
(60) Provisional application No. 61/681,918, filed on Aug. 10, 2012.

(51) Int. Cl. G01N 33/58; C12Q 1/68; G01N 33/573

(52) U.S. Cl. CPC G01N 33/582; G01N 33/573; C12Q 1/6886; C12Q 2600/16; C12Q 2600/156; C12Q 2600/106; G01N 2333/914; G01N 2333/916

(57) ABSTRACT

Bacillus Calmette-Guerin (BCG) administration plays a central role in managing carcinoma in situ of the bladder. Unfortunately, recurrence or progression of disease is seen in up to 30% of treated patients. Disclosed herein is a method for predicting responsiveness to treatment with BCG based on BCG internalization by bladder cancer cells, the presence or absence of mutations associated with BCG uptake or a combination thereof.

### CROSS REFERENCE TO RELATED APPLICATIONS

This application claims the priority of U.S. provisional application No. 61/681,918 filed Aug. 10, 2012, the entire disclosure of which is incorporated herein by reference.

### REFERENCE TO SEQUENCE LISTING

This application contains a Sequence Listing, created on Jul. 30, 2013; the file, in ASCII format, is designated 3314023AWO_Sequence Listing_ST25.txt and is 1.52 kilobytes in size. The sequence listing file is hereby incorporated by reference in its entirety into the application.

### FIELD OF THE INVENTION

The present invention relates generally to treatment of bladder cancer and in particular to targeted therapy for bladder cancer patients based on a prospective assessment of the sensitivity of bladder cancer cells obtained from the patient to a therapeutic agent, bacillus Calmette Guerin (BCG).

### BACKGROUND OF THE INVENTION

Bladder cancer is among the most common tumors diagnosed in the United States, with an estimated annual incidence of 70,530 new cases and 14,680 deaths in 2010 (1). Approximately 70% of bladder tumors are classified as superficial (non-muscle-invasive). Treatment of superficial bladder cancer by transurethral resection alone is associated with a 40-80% risk of recurrence and a 10-27% chance of progressing to muscle-invasive, regional or metastatic disease (2).

Bacillus Calmette-Guerin (BCG) is a therapeutic agent approved by the US Food and Drug Administration as a primary therapy of carcinoma in situ (CIS) of the bladder. BCG is an attenuated strain of Mycobacterium bovis that was derived by prolonged in vitro passage of virulent M. bovis at the Pasteur Institute in the early 1900s.

For bladder cancer, patients typically receive repeated instillations of live bacteria into the bladder. BCG administration plays a central role in managing CIS as well as high grade Ta (papillary) and T1 (lamina propria invasive) lesions after transurethral resection (3). BCG treatment is the most effective agent to decrease cancer recurrences in superficial bladder cancer. However, up to 30% of treated patients experience recurrence or progression of disease (3).

The present invention arises from the need for a prognostic indicator of BCG sensitivity in bladder cancer, one that can help tailor bladder cancer treatment for individual patients based on a prospective assessment of their responsiveness to BCG.

### SUMMARY OF THE INVENTION

The present invention relates to a method for the prospective identification of bladder cancer patients who likely would be responsive to treatment with BCG. The method involves genotypic and phenotypic characteristics of bladder cancer cells from the patient which allow the clinician to differentiate between bladder cancer patients who will likely respond to treatment with BCG and those who are likely to be unresponsive or refractory to treatment with BCG, allowing decisions to be made early with respect to appropriate treatment for all patients.

In one aspect, the invention relates to a method for determining the responsiveness of a bladder cancer patient to treatment with bacillus Calmette Guerin (BCG), the method comprising (a) contacting an isolated bladder cancer cell or cells from the patient with BCG containing a detectable label for a period of time sufficient for said BCG to be internalized by said cell(s); (b) determining the amount of BCG uptake by said isolated bladder cancer cell(s); (c) comparing the amount of BCG uptake by said isolated bladder cancer cell(s) with a reference amount of BCG uptake by normal urothelial cells or with a reference amount of BCG uptake in known BCG permissive cells; and (d) determining that the patient will be responsive to therapy with BCG when the amount of labeled BCG uptake by said isolated bladder cancer cell is greater than the amount of uptake by normal urothelial cells or equal to or greater than the uptake by known BCG-sensitive cells. BCG used in the present method comprises a detectable label, for example, BCG that has been transformed to express a fluorescent protein such as green fluorescent protein (GFP) or mCherry. BCG uptake by the cells can be readily monitored using flow cytometry and/or confocal microscopy to assess the amount of fluorescence associated with the cells.

In a related aspect, the invention relates to a method for selecting treatment options for a patient with bladder cancer, the method comprising (a) contacting an isolated bladder cancer cell or cells from the patient with BCG containing a detectable label for a period of time sufficient for said BCG to be internalized by said cell(s); (b) determining the amount of BCG uptake by said isolated bladder cancer cell(s); and (c) comparing the amount of BCG uptake by said isolated bladder cancer cell(s) with a reference amount of BCG uptake by normal urothelial cells or a reference amount of BCG uptake by know BCG permissive cells, wherein treatment with BCG is indicated when BCG uptake by said isolated bladder cancer cell(s) from the patient is greater than BCG uptake by normal urothelial cells or equal to or greater than the BCG uptake by known BCG permissive cells. BCG uptake by the cells can be determined using flow cytometry and/or confocal microscopy to assess the amount of fluorescence associated with the cells.

In another aspect, the invention relates to a method for determining responsiveness of a bladder cancer patient to treatment with BCG, the method comprising obtaining a bladder cancer cell or cells from the patient and determining the presence in said cell(s) of one of (a) a RAS-activating mutation, (b) decreased expression or deletion of PTEN, (c) overexpression of Pak1, or (d) elevated expression of Cdc42 compared to the level of Cdc42 expression in normal urothelial cells, wherein the presence of at least one of (a)-(d) indicates responsiveness to treatment with BCG. Ras-activating mutations include all H-Ras, K-Ras and N-Ras activating mutations, including but not limited to those, for example, in codon 12 of H-Ras (G12V) and K-Ras (G12C).

In yet another aspect, the invention relates to a kit for assessing BCG uptake by a patient's bladder cancer cells that includes BCG comprising a detectable label and a cell or a panel of cells that are known responders. The kit may further include BCG resistant cells that are known to exhibit poor BCG uptake as a control.

In another aspect, the invention relates to a method for identifying an agent that enhances BCG uptake by bladder cancer cells, the method comprising: (a) contacting a known resistant bladder cancer cell with a test agent; (b) contacting said known resistant bladder cancer cell with BCG containing a detectable label for a period of time sufficient for BCG to be internalized by the permissive cell(s); (c) determining the amount of BCG uptake by said known resistant bladder cancer cell; (d) comparing the amount of BCG uptake by said known resistant bladder cancer cell with (i) a reference amount of BCG uptake by normal bladder cells; (ii) a reference amount of BCG uptake by known BCG-permissive cells; and/or (iii) the amount of BCG uptake in the resistant cell prior to exposure to the test agent; (e) determining that the agent tested enhances BCG uptake by bladder cancer cells when the amount of BCG uptake in said cell is equal to or greater than the reference amount of BCG uptake by known BCG-permissive cells or greater than the amount of BCG uptake in normal cells or resistant cells that have not been exposed to the agent.

### BRIEF DESCRIPTION OF THE DRAWINGS

FIG. 1 shows heterogeneous BCG susceptibility among bladder cancer lines. A. The bladder cancer cell lines J82, T24, UM-UC-3, MGH-U3, MGH-U4, and VMCUB-3 were incubated with BCG-GFP (MOI 10:1) for 4 hrs. At the end of the incubation period the cells were washed, detached, and evaluated by flow-cytometry. For each cell line, representative flow-plots of uninfected cells (left panel) and infected cells (right panel) are shown. In each flow-plot X-axis measures green fluorescent protein (GFP)-fluorescence intensity and Y-axis measures pacific-blue fluorescence intensity (empty channel used to facilitate gating due to auto-fluorescence of the cells). Number within the gate represents percentage of GFP-positive events out of total events. B. The specified cell lines were incubated with BCG-GFP for the specified time periods and BCG uptake measured by flow-cytometry. Graphs show percents of cells that have taken up BCG at 4 hours (left panel) and at 24 hours (right panel). The data corresponds to the mean of three independent experiments±SEM. C. The specified cell lines were incubated with BCG-GFP for 24 hours and evaluated by confocal microscopy. Nuclei are stained with Hoechst (blue), actin with Texas-red phalloidin (red) and GFP-expressing BCG is shown in green. Top: 20× magnification of the specified cell lines. Scale bar is 50 μm. Bottom: 63× magnification of the cell lines T24 and UM-UC-3 infected with BCG-GFP. Scale bar is 15 μm. Data are representative of two independent experiments. D. The bladder cancer cell lines J82, T24, UM-UC-3, MGH-U3, MGH-U4, and VMCUB-3 were incubated with BCG-GFP (MOI 10:1) for 4 or 24 hrs. At the end of incubation cells were stained with pacific-blue labelled annexin V (marker of apoptosis) and evaluated by flow-cytometry. The data corresponds to the mean of three independent experiments±SEM.

FIG. 2 shows the effect of small-molecule inhibitors on BCG uptake by bladder cancer cells. The cell lines J82, T24, and UM-UC-3 were pretreated for one hour with the specified small molecule inhibitors at the stated concentration. BCG-GFP was then added, and incubated with the cells for 4 hours in the presence of the inhibitors. At the end of the incubation period the cells were washed, and BCG uptake measured by flow-cytometry. For each inhibitor, the percent of cells infected by BCG-GFP is shown as compared with percent of infected cells in the presence of DMSO (vehicle control). The data corresponds to the mean of three independent experiments±SEM. *, P<0.05; **, P<0.005; ***, P<0.0005 compared with DMSO

FIG. 3 illustrates the role of the Rac1-Cdc42-Pak1 pathway in BCG uptake by bladder cancer cells. A. The cell lines J82, T24, and UM-UC-3 were pretreated with the small-molecule inhibitors IPA-3 (an inhibitor of Pak1) or Y-27632 (an inhibitor of RhoA Kinase) at the stated concentrations. After 1 hour BCG-GFP was added, and incubated with the cells for 4 hours in the presence of the inhibitors. At the end of the incubation period the cells were washed, and BCG uptake was measured by flow-cytometry. For each inhibitor, the percent of cells infected by BCG-GFP is shown as compared with percent of infected cells in the presence of DMSO (vehicle control). The data corresponds to the mean of three independent experiments±SEM. *, P<0.05; **, P<0.005 compared with DMSO. B. J82, T24, and UM-UC-3 were stably transfected with empty vector or with vectors expressing DN-Rac1 (T17N) or DN-Cdc42 (T17N) with an N-terminal myc-tag. Cells were incubated with BCG-GFP for 4 hours, and BCG uptake measured by flow-cytometry. Expression of myc-tagged Rac1 (T17N) or myc-tagged Cdc42 (T17N) was demonstrated by western blotting. The data corresponds to the mean of three independent experiments±SEM. *, P<0.05; **, P<0.005; ***, P<0.0005. C. UM-UC-3 was stably transfected with non-targeting or two forms of Pak1 shRNA. Cells were incubated with BCG-GFP for 4 hours, and uptake of the BCG was measured by flow-cytometry. Knock-down of Pak1 in the Pak1 shRNA transformed cells was demonstrated by Western blotting. The data corresponds to the mean of three independent experiments±SEM. ***, P<0.0005. D. The cell lines J82, T24, and UM-UC-3 were stably transfected with an empty vector, vector expressing N-terminal myc-tagged wild-type Pak1, or vector expressing N-terminal myc-tagged Pak1 (K299R) (a dominant-negative form of Pak1). The cells were incubated with BCG-GFP for 4 hours, and BCG uptake was measured by flow-cytometry. Expression of myc-tagged wild-type Pak1 and Pak1 (K299R) was demonstrated by western blotting. The data corresponds to the mean of three independent experiments±SEM. *, P<0.05; **, P<0.005; ***, P<0.0005.

FIG. 4 illustrates that BCG uptake is independent of dynamin and clathrin. A. T24 and UM-UC-3 were transiently transfected with empty vector or with GFP-tagged dynamin 2 (aa) wild type or GFP-tagged dynamin 2 (aa) (K44A) (a dominant-negative form of dynamin). 24 hours after transfection the cells were washed and infected with BCG-mCherry at an MOI of 10:1. Uptake of BCG by cells expressing the GFP-tagged protein was measured after 24 hours using flow-cytometry. The data corresponds to the mean of three independent experiments±SEM. B. MGH-U4 was transiently transfected with empty vector or with GFP-tagged dynamin 2 (aa) wild type or GFP-tagged dynamin 2 (aa) (K44A). 24 hours after transfection the cells were washed and infected with BCG-mCherry at an MOI of 10:1. Uptake of BCG by cells containing the GFP-tagged protein was measured after 24 hours using flow-cytometry. The data corresponds to the mean of three independent experiments±SEM. C. T24 and UM-UC-3 were stably transduced with lentiviruses bearing non-targeting or three shRNAs targeting the clathrin heavy chain. Cells were incubated with BCG-GFP for 4 hours, and uptake of BCG was measured by flow-cytometry. Knock-down of clathrin heavy-chain by clathrin heavy-chain shRNA was demonstrated by Western blotting. The data corresponds to the mean of three independent experiments±SEM

FIG. 5 illustrates the co-localization of fluid-phase with BCG. Confocal microscopy of T24 and UM-UC-3 incubated with BCG-GFP for 4 hours in the presence of red-fluorescent dextran (MW 10,000) in the media. BCG-GFP is shown in green, and red-fluorescent dextran within the fluid phase is shown in red. Arrows point to location of BCG. Scale bar is 15 μm. Data are representative of two independent experiments.

FIG. 6 illustrates the role of the PTEN/PI3K/Akt pathway in BCG uptake by bladder cancer cells. A. Western blot of J82, T24, UM-UC-3, MGH-U3, MGH-U4, and VMCUB-3. Expression of PTEN, Akt phosphorylated at serine 473, total Akt, and β-actin (loading control) were evaluated. Data are representative of three independent experiments. B. The cell lines J82, T24, and UM-UC-3 were pretreated with the small-molecule inhibitors wortmannin, Akti XIII or rapamycin at the stated concentrations. After 1 hour BCG-GFP was added, and incubated with the cells for 4 hours in the presence of the inhibitors. At the end of the incubation period the cells were washed, and BCG uptake was measured by flow-cytometry. For each inhibitor, the percent of cells infected by BCG-GFP is shown as compared with percent of infected cells in the presence of DMSO (vehicle control). On left, Western blotting of UM-UC-3 after treatment for 1 hour with DMSO (control), wortmannin, Akti XIII, or rapamycin. Expression of Akt phosphorylated at serine 473, total Akt, S6 kinase phosphorylated at threonine 389, total S6 kinase, and β-actin (loading control) were evaluated. The data corresponds to the mean of three independent experiments±SEM. *, P<0.05; **, P<0.005 compared with DMSO. C. The cell lines J82, T24, and UM-UC-3 were stably transfected with empty vector, lipid phosphatase inactive PTEN mutant (PTEN C124S) or wild-type PTEN. Cells were incubated with BCG-GFP for 24 hours, and BCG uptake measured by flow-cytometry. Expression of the PTEN-expressing vectors was demonstrated by Western blotting. The data corresponds to the mean of three independent experiments±SEM. **, P<0.005; ***, P<0.0005. D. The cell lines MGH-U3 and VMCUB-3 were stably transfected with non-targeting or PTEN shRNA, incubated with BCG-GFP for 24 hours, and BCG uptake measured by flow-cytometry. Knock-down of PTEN by PTEN shRNA was demonstrated by Western blotting. The data corresponds to the mean of three independent experiments±SEM. **, P<0.005. E. MGH-U4 transfected with PTEN shRNA was pretreated with DMSO or IPA-3 at the specified concentrations for 1 hour and incubated with BCG-GFP for 4 hours in the presence of the inhibitor. BCG uptake was measured by flow-cytometry and compared to MGH-U4 transfected with non-targeting shRNA and treated with DMSO. The data corresponds to the mean of three independent experiments±SEM

FIG. 7 shows that activated Ras stimulates BCG uptake via macropinocytosis. A. The cell lines MGH-U3, MGH-U4, and VMCUB-3 were stably transfected with an empty vector, or the activated Ras forms K-ras (G12D) (top panel) or H-ras (G12V) (bottom panel). The cells were incubated with BCG-GFP for 4 hours, and BCG uptake measured by flow-cytometry. The data corresponds to the mean of three independent experiments±SEM. B. Phase-contrast and fluorescence microscopy of the cell line VMCUB-3 transfected with an empty vector, K-ras (G12D) or H-ras (G12V), and infected with BCG-GFP for 24 hours. BCG-GFP is shown in green. Scale bar is 25 μm. Data are representative of two independent experiments. C. Confocal microscopy of VMCUB-3 transfected with an empty vector or K-ras (G12D). Cells were incubated with BCG-GFP for 3 hours in the presence of red-fluorescent dextran (MW 10,000) in the media. BCG-GFP is shown in green, and red-fluorescent dextran within the fluid phase is shown in red. Arrows point to location of BCG. Scale bar is 15 μm. Data are representative of two independent experiments. D. VMCUB-3 transfected with H-ras (G12V) was pretreated with DMSO, IPA-3 or wortmannin at the specified concentrations for 1 hour, and incubated with BCG-GFP for 4 hours in the presence of the inhibitor. BCG uptake was measured by flow-cytometry and compared to VMCUB-3 transfected with an empty vector and treated with DMSO. The data corresponds to the mean of three independent experiments±SEM.

FIG. 8 is representative flow cytometry analysis showing the gating strategy to determine the percent of BCG-GFP infected cells. Cells were pre-gated in an FSC/SSC scattergram. scattergram. Because of auto-fluorescence in the cell lines used, an empty channel (Pacific blue) was used to facilitate discrimination of GFP-positive events. In each experiment uninfected cells were used as a control to optimize gating.

FIG. 9 shows the effect of small molecule inhibitors on the uptake of fixed BCG. UM-UC-3 was pretreated for one hour with the specified small molecule inhibitors at the stated concentration. BCG-GFP was fixed in 4% PFA, washed twice, and added to the cells for 4 hours in the presence of the inhibitors. At the end of the incubation period the cells were washed, and BCG uptake measured by flow cytometry. For each inhibitor, the percent of cells infected by BCG-GFP is shown as compared with percent of infected cells in the presence of DMSO (vehicle control). Killing of the BCG by the fixative was confirmed by plating the fixed BCG on 7H10 plates and observing no colonies.

FIG. 10 shows the uptake of a non-pathogenic mycobacterium. The cell lines J82, T24, UM-UC-3, MGH-U3, MGH-U4, and VMCUB-3 were incubated with GFP-expressing M. smegmatis (MOI 10:1) for 4 hrs. At the end of the incubation period uptake of M. smegmatis was measured by flow cytometry. The data corresponds to the mean of three independent experiments±SEM.

FIG. 11 shows the effect of dynamin constructs and clathrin shRNA on uptake of fluorescent transferrin. A T24 was transiently transfected with empty vector or with GFP-tagged dynamin 2 (aa) wild type or GFP-tagged dynamin 2 (aa) (K44A). 24 hours after transfection the cells were incubated with fluorescent transferrin for 15 minutes and uptake was measured by flow cytometery. Shown is the mean Alexa 568 fluorescence for each sample. The data corresponds to the mean of three independent experiments±SEM. B T24 was stably transduced with lentiviruses bearing non-targeting or three shRNAs targeting the clathrin heavy chain. Cells were incubated with fluorescent transferrin for 15 minutes and uptake was measured by flow cytometry. Shown is the mean Alexa 568 fluorescence for each sample. The data corresponds to the mean of three independent experiments±SEM.

FIG. 12 shows representative images of BCG uptake in bladder cancer cells. Patient specimens #13 and #16, and bladder cancer cell line controls MGHU4 (BCG-resistant) and UMUC3 (BCG-sensitive) were infected with GFP-expressing BCG for 24 hours.

### DETAILED DESCRIPTION OF THE INVENTION

All publications, patents and other references cited herein are incorporated by reference in their entirety into the present disclosure.

In practicing the present invention, many conventional techniques in microbiology, cell biology and molecular biology are used, which are within the skill of the ordinary artisan. Some techniques are described in greater detail in, for example, Molecular Cloning: a Laboratory Manual 3rd edition, J. F. Sambrook and D. W. Russell, ed. Cold Spring Harbor Laboratory Press 2001, the contents of this and other references containing standard protocols, known to and relied upon by those of skill in the art, including manufacturers' instructions are hereby incorporated by reference as part of the present disclosure.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Abbreviations used herein:

AKTI: Akti XIII

BCG: Bacillus Calmette-Guerin

BLEB: Blebbistatin

Cdc42: cell division cycle 42

CYTO: Cytochalasin D

DMSO: Dimethyl sulfoxide

EIPA: 5-(N-Ethyl-N-isopropyl) amiloride

GENI: Genistein

PTEN: phosphatase and tensin homolog

RAPA: Rapamycin

STAU: Staurosporine

WORT: Wortmannin.

As used herein, “cancer” refers to cells or tissues that have characteristics such as uncontrolled proliferation, immortality, metastatic potential, increased anti-apoptotic activity, etc.

As used herein, a “subject” refers to any animal (e.g. a mammal), including, but not limited to, humans, non-human primates, companion animals, rodents, and the like. Typically, the terms “subject” and “patient” are used interchangeably herein, particularly in reference to a human subject.

As used herein, “responsiveness” refers to the development of a favorable response when a cell or subject is contacted with an agent (e.g. a therapeutic agent.) By way of non-limiting example, a favorable response can be inhibition of cell growth when a cell is contacted with a particular agent and an unfavorable response can be the accelerated growth of a tumor when a patient with a tumor is contacted with a particular agent.

As used herein, “agent” refers to a substance that elicits a response from a cell or subject when said cell or subject is contacted with an agent. An agent can be a small molecule, a peptide, an antibody, a natural product, a nucleic acid, etc. In some cases, an agent can be a composition used in the treatment of, or used to treat, a subject. An “inhibitor” is an agent that interferes with the normal function or effect of a polypeptide, cell, subject, etc.

As used herein, “inhibition” or “to inhibit” means to reduce a function of a polypeptide, cell or subject in response to an agent (e.g. an inhibitor) relative to such function of said polypeptide, cell or subject in the absence of such agent.

As used herein, “enhancement” or “to enhance” means to increase a response or effect, for example, of a polypeptide, cell or subject in response to an agent relative to the ordinary response or effect of said polypeptide, cell or subject in the absence of such agent.

As used herein, “treatment” or to “treat” means to address a disease in a subject and includes preventing the disease, delaying the onset of disease, delaying the progression of the disease, eradicating the disease (e.g. causing regression of the disease), etc.

The term “predicting responsiveness to treatment with BCG”, as used herein, is intended to refer to an ability to assess the likelihood that treatment of a subject with BCG will or will not be effective in (e.g., provide a measurable benefit to) the subject. In particular, such an ability to assess the likelihood that treatment will or will not be effective typically is exercised before treatment with BCG is begun in the subject. However, it is also possible that such an ability to assess the likelihood that treatment will or will not be effective can be exercised after treatment has begun but before an indicator of effectiveness (e.g., an indicator of measurable benefit) has been observed in the subject or when progression of the disease is evident after an initial period of responsiveness.

As used herein, “sensitive” or “permissive” refers to the ability to respond to an agent; in the present disclosure, it refers to the ability of a patient with bladder cancer or of the bladder cancer cells themselves to respond to treatment with BCG.

As used herein, “resistance” or “resistant” refers to a lack of response by a cell to an agent to which the cell may have responded previously (e.g. the cell is “resistant to” such agent). In the context of a patient, “resistance” refers to lack of response of a patient to an agent to which said patient used to respond. Resistance can be acquired (e.g. develops over time) or inherent or de novo (e.g. a cell or subject never responds to an agent to which other similar cells or subjects would respond). By way of non-limiting example, a subject is said to be resistant to treatment when such subject no longer responds to such treatment (e.g. the treatment of a subject with an agent results in initial delay of disease progression, but then such disease progresses even if said subject is still treated with such agent.)

### Mechanism of BCG Uptake

Epithelial cells are not the usual target of mycobacteria; the main cell type involved in M. tuberculosis infection is the macrophage, and the receptors utilized by macrophages for phagocytosis of M. tuberculosis have been comprehensively described (41). Disclosed herein is a novel mechanism underlying BCG uptake within epithelial cells, which is dependent on the actin cytoskeleton, inhibited by EIPA, and controlled by Cdc42, Rac1 and Pak1. Inhibition of dynamin or clathrin did not inhibit BCG uptake. Perhaps most importantly, BCG was taken up with fluid phase markers. Overall, these features are most consistent with uptake by macropinocytosis. Intriguingly, some characteristics of BCG uptake by bladder cancer cells are similar to uptake of Uropathogenic Escherichia coli (UPEC) by the bladder epithelium. UPEC invasion of bladder epithelial cells is dependent on Cdc42 and PI3K activation through activation of Rac1 (42). However, one major difference is that UPEC actively triggers these pathways through secretion of cytotoxic necrotizing factor-1 (CNF1), which activates Rac1 (43), while BCG appears to act as an “innocent bystander”, relying on oncogenic activation of these pathways to gain entry into the cells. A second difference is that UPEC entry is dependent on dynamin 2 (44) and clathrin (45), while BCG uptake is independent of both of these factors.

Others have shown that BCG attachment and uptake by bladder cancer cells is facilitated through attachment of BCG fibronectin attachment protein (FAP) to fibronectin on bladder cancer cells (46). Receptor-mediated uptake of large particles, via phagocytosis, or by the clathrin-dependent pathway that is utilized for uptake of Listeria, is typically dependent on dynamin (10, 24, 25). BCG uptake, however, was not dependent on dynamin. One explanation for this apparent discrepancy is that uptake of BCG does not occur through a classical receptor-mediated uptake pathway. Rather, BCG that is either adjacent to bladder cancer cells or attached to them through a receptor is internalized because of increased membrane ruffling that accompanies macropinocytosis. The presence of a receptor for BCG attachment, such as a5β1 integrin, would lead to more mycobacteria being in intimate contact with the cells, and would thus promote this process, resulting in increased uptake of BCG. Although macropinocytosis has traditionally been described as receptor-independent (21), there have been some recent reports describing receptor-dependent pathways of macropinocytosis in the uptake of some viruses (47, 48).

To date, no independent prognostic factor for bladder tumor response to BCG has been identified. Despite over 30 years of clinical experience with intravesical BCG for bladder cancer, its mechanism of antitumor effect remains unknown and no markers exist to predict which patients will respond to therapy. Direct and indirect immune mechanisms have been hypothesized to play a role in BCG's antitumor effect (4), as have direct cytotoxic effects on the tumor cell (5). Whatever the eventual mechanism of toxicity to bladder cancer cells, it does seem clear that BCG attachment to tumor cells, leading to internalization and processing of the mycobacterium, plays a crucial role in activation of BCG mediated anti-tumor activity (6-8).

The present disclosure provides a method for determining whether a subject in whom bladder cancer has been diagnosed will be responsive to treatment with bacillus Calmette Guerin (BCG). The method provides a mechanism for guiding treatment options early on.

The method is based on the observation that bladder cancer cell lines vary considerably in their propensity to take up BCG. It was found that this property is dependent on activation of several oncogenic signaling pathways, resulting in increased macropinocytosis and uptake of BCG.

It turns out that the same pathways involved in bladder cancer oncogenesis also determine BCG uptake. Alterations in the PTEN/PI3K/Akt pathway are frequently present in human bladder cancers. These include decreased expression or deletion of the tumor suppressor PTEN, activating mutations of PI3K, and, rarely, activating mutations of Akt1 (35). A sizeable fraction of bladder cancers harbor activating mutations of Ras, most commonly H-ras mutations (36). Cdc42 also appears to have a role in bladder cancer; expression of Cdc42 has been shown to be higher in bladder cancer compared to normal urothelial cells, and RNA interference of Cdc42 was found to suppress growth of bladder cancer cells (37, 38). Pak1 has been found to be overexpressed in a large proportion of bladder cancers (39), and may also be a marker of recurrence after transurethral resection of superficial bladder cancer (40).

The pathways determining BCG uptake by bladder cancer cells, namely, PTEN-PI3K, Ras, and Cdc42-Rac1-Pak1, are known to be interconnected. The oncoprotein Ras can activate PI3K (31), and is also able to activate Rac1 through its action on the guanine nucleotide exchange factor (GEF) Tiam1 (32). Rac1 can also be activated by increased phosphatidylinositol (3,4,5)-triphosphate (PIP3) concentrations (33), which would be expected to occur through PI3K activation or through PTEN loss. Cdc42 can itself activate PI3K (34).

The findings disclosed herein possibly explain why treatment with BCG is successful in most, but not all, patients with bladder cancer. BCG therapy would be expected to provide the most benefit for those patients whose cancer contains mutations activating the pathways of BCG uptake, such as decreased PTEN expression or activating Ras mutations. These findings could also provide a mechanism of specificity for BCG infection of tumor cell compared to the normal urothelium, which does not contain mutations activating BCG uptake.

### Prognostic Determination of BCG Responsiveness in Non-Invasive Bladder Cancer Patients

Internalized BCG can be identified within urothelial cells in bladder washings of patients treated with BCG. Accordingly, in one embodiment, the method of the invention involves assessing the ability of isolated bladder cancer cells to take up BCG using an in vitro system of infection that employs a BCG having a detectable label. One or more bladder cancer cells are obtained from a subject either from a urine sample, bladder washings or biopsy of a bladder tumor. In some instances, a method to enrich cancer cells in a urine or bladder washing sample may be desirable.

The cells are then contacted with “labeled” BCG at a multiplicity of infection (MOI) of about 2:1 to 20:1; in one embodiment, an MOI of about 10:1 is used. BCG for use in practicing the present invention is labeled with a detectable marker. In one embodiment, the BCG are transformed so that they express detectable levels of a fluorescent protein marker, for example, green fluorescent protein. The cells are contacted with the labeled BCG for a time sufficient for uptake of the BCG to occur, for example, between 1 to 48 hours; in one embodiment from 12 to 36 hours; in one embodiment from 18 to 24 hours.

Once sufficient time for BCG uptake by the bladder cancer cells has elapsed, the cells are assessed for uptake using flow cytometry and/or confocal microscopy in accordance with methods known to those of skill in the art. Uptake in the sample cells is then compared to uptake in known BCG-permissive cells, for example UM-UC-3 cells or T24 cells (catalog nos: CRL1749 and HTB-4, respectively, American Type Tissue Collection, Manassas Va.). In some embodiments, comparison of uptake in patient cells to uptake in normal urothelial cells may be desired. Patient cells having uptake equal to or greater than the uptake of known BCG-permissive cells indicate that the patient cells are permissive and that the patient will be responsive to therapy with BCG. In some instances, BCG infection of about 10% of the bladder cancer cells or greater indicates permissiveness/responsiveness.

In another embodiment, bladder cancer cells are obtained from a patient and assessed for the presence of one or more of (a) decreased expression or deletion of PTEN; (b) an activating mutation of Ras (K-Ras, H-Ras or N-Ras, for example a mutation at codon 12 of Ras such as H-Ras (G12V) or K-Ras (G12C); (c) overexpression of Pak1; or (d) elevated expression of Cdc42 compared to the level of Cdc42 expression in normal urothelial cells. Ras proteins normally act as signaling switches, which alternate between the active and inactive states. Somatic point mutations in codons 12, 13 and 61 of the N-Ras and K-Ras genes, for example, occur in many malignancies, resulting in persistently active forms of the protein. For purposes of practicing the method of the present invention, Ras-activating mutations include all H-Ras, K-Ras and N-Ras activating mutations, including but not limited to those, for example, in codon 12 of H-Ras (G12V) and K-Ras (G12C). Methods that can be used to identify mutations in the isolated bladder cancer cell(s) are well known in the art and include by way of example Western Blotting, Real-time polymerase chain reaction (RT-PCR), DNA microarray technology, Nanostring Technology, and Sanger sequencing or high-throughput sequencing, such as Illumina Sequencing.

Screening for Agents that Enhance BCG Uptake

Having identified BCG uptake as a seminal event in responsiveness to BCG treatment, the disclosed method can be further exploited to identify agents that can be used to enhance BCG uptake by resistant bladder cancer cells. Bladder cancer cells that are known to be resistant to BCG are exposed to an agent prior to or contemporaneously with exposure to BCG. Uptake in the cells is then compared to the BCG uptake in normal cells, known permissive cells and resistant cells that have not been exposed to the test agent to determine whether the agent promotes uptake in the resistant cell. Likely candidates for agents that promote BCG uptake are those which are involved in the activation of the PI3K or Ras pathways.

### Kits

Kits for assessing BCG uptake by a patient's bladder cancer cells is encompassed by the present invention. A kit includes (1) BCG comprising a detectable label and (2) a cell or a panel of cells that are known responders. The kit may further include BCG resistant cells that are known to exhibit poor BCG uptake for comparison.

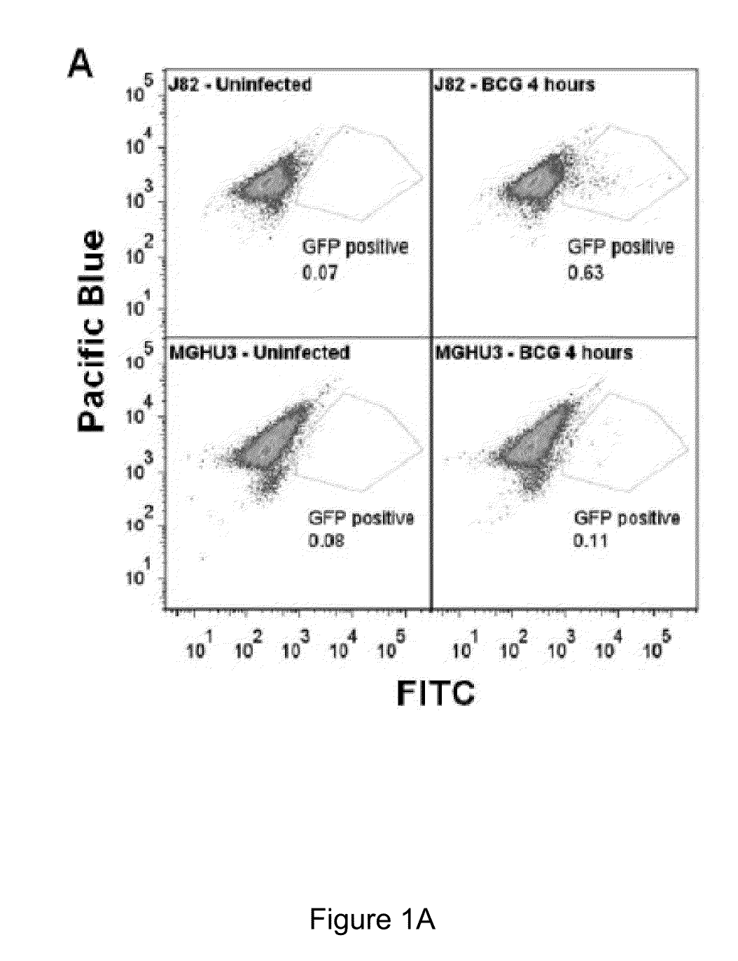

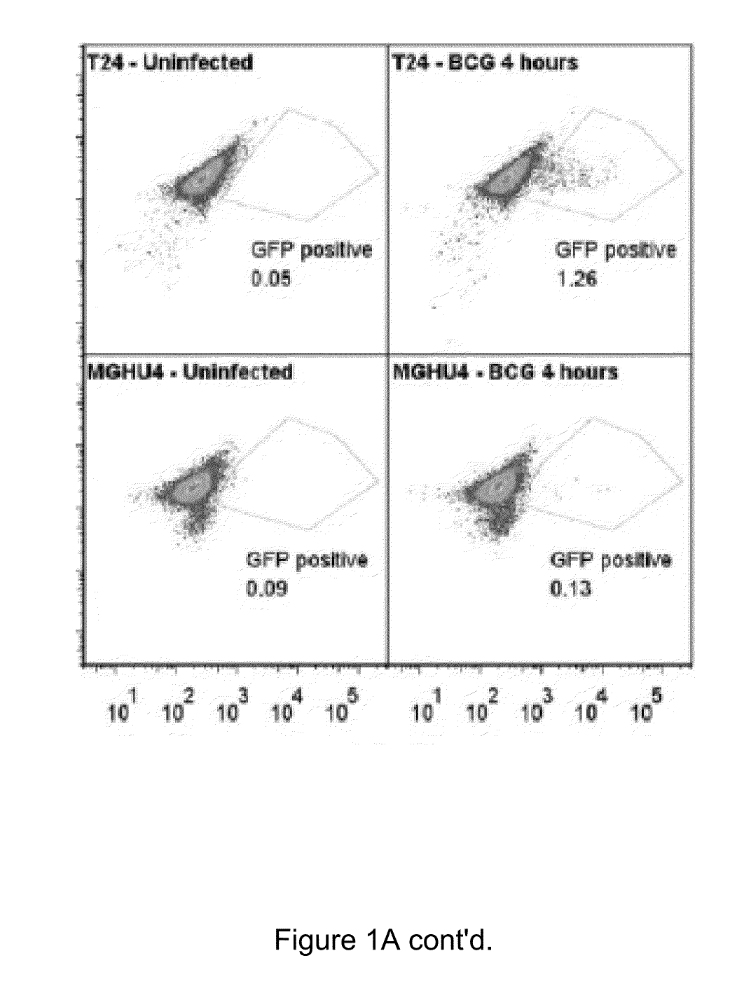

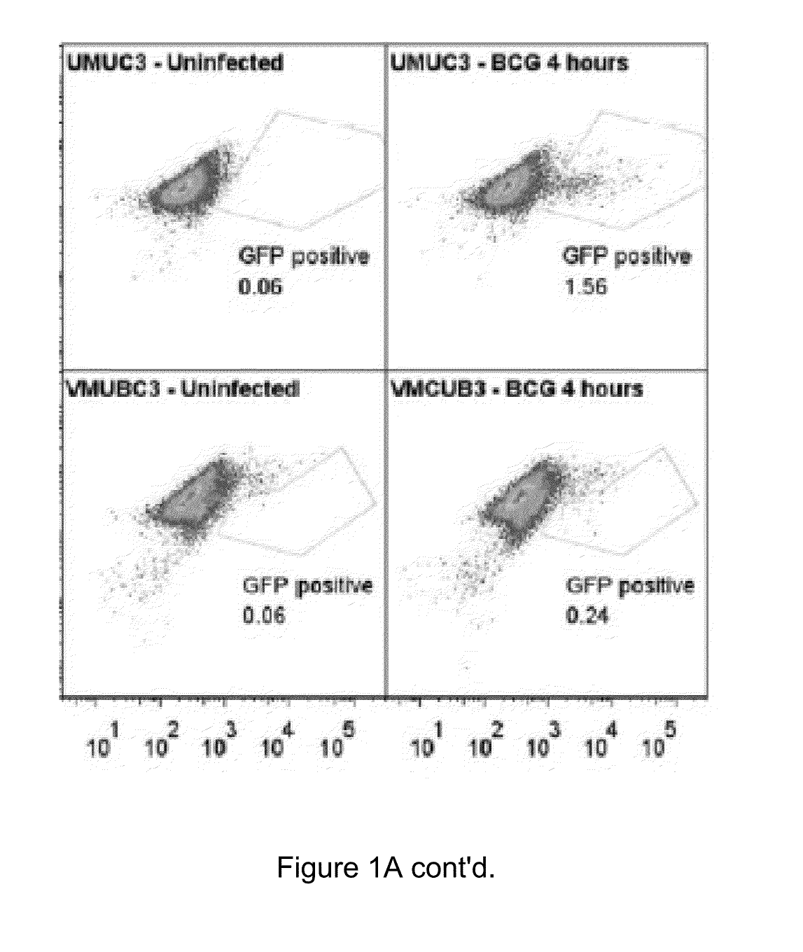

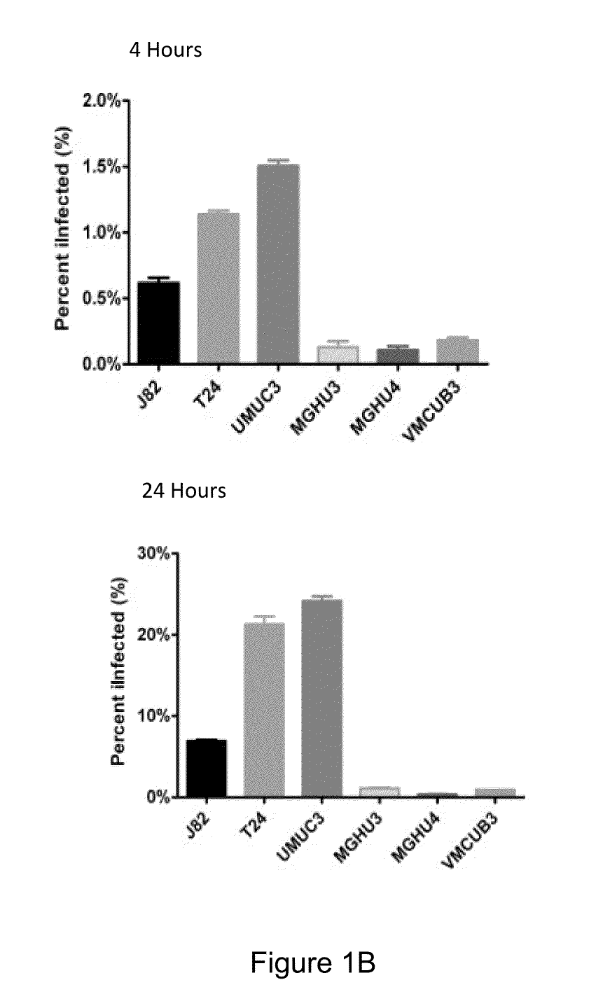

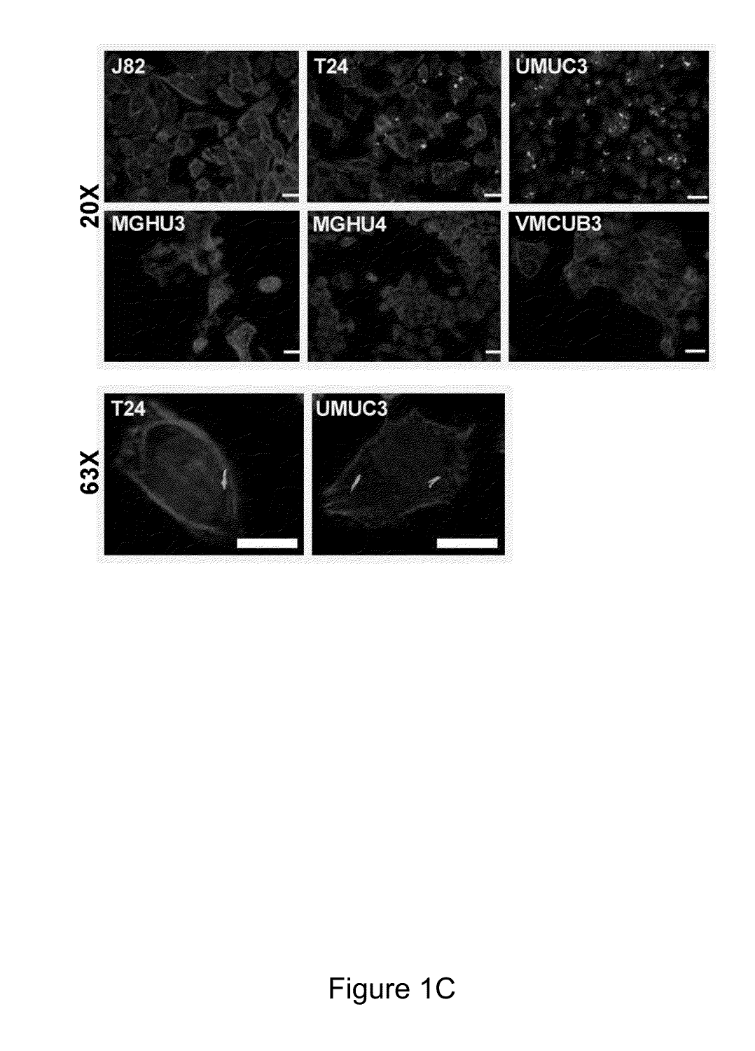

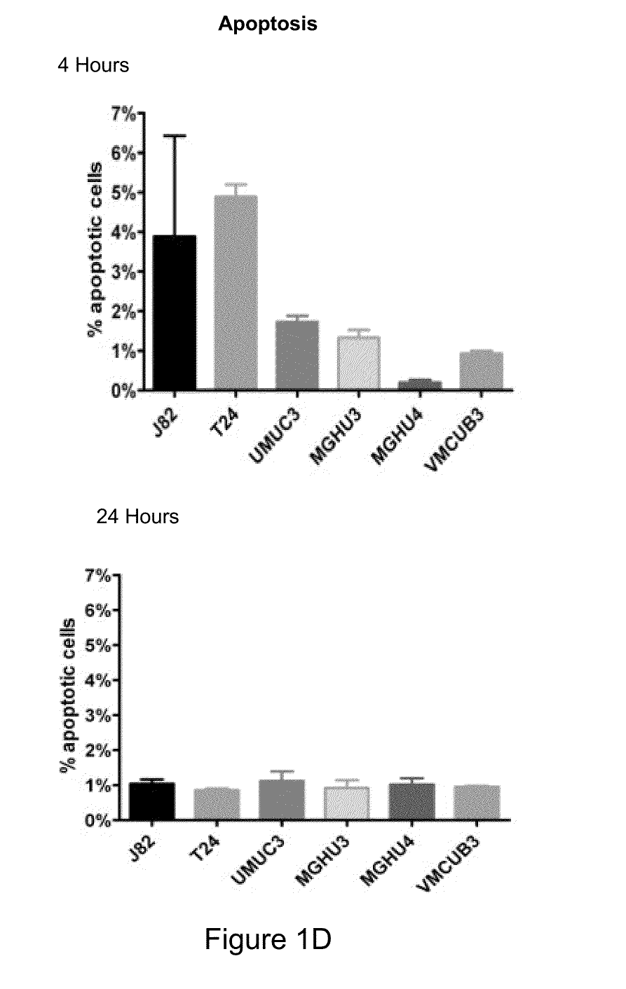

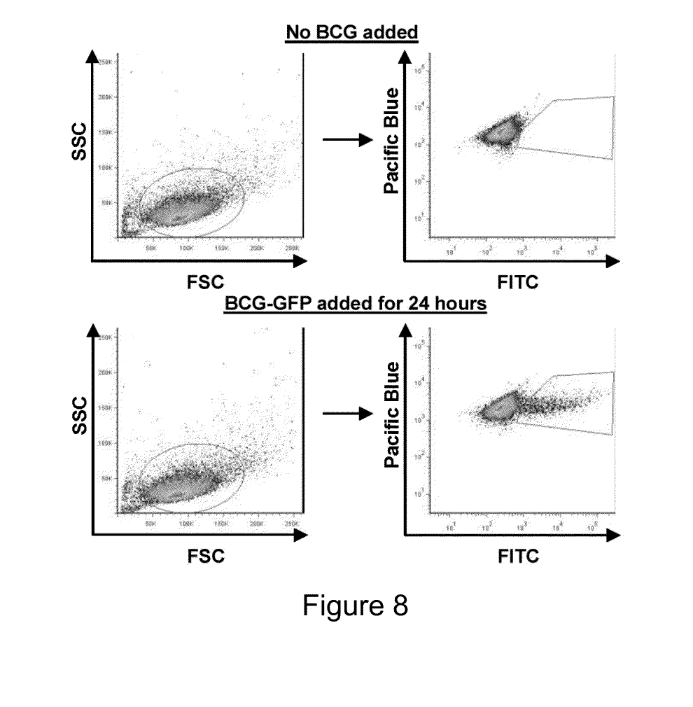

To study the mechanism of BCG infection of bladder cancer cells, an in vitro system of infection was designed using a BCG strain expressing Green Fluorescent Protein (GFP) and a panel of bladder cancer cell lines derived from human tumors. Uptake of BCG was monitored by flow cytometry and/or confocal microscopy. A panel of six bladder cancer cell lines was assembled and it was first asked whether they differ in their susceptibility to BCG infection. The bladder cancer cell lines J82, T24, UM-UC-3, MGH-U3, MGH-U4, and VMCUB-3 were infected with BCG-GFP, and uptake of BCG was determined using flow-cytometry (FIGS. 1A, 8). The cell lines could be categorized into two groups according to their susceptibility to BCG infection; three of the cell lines (UM-UC-3, T24 and to a lesser degree J82) readily took up BCG, with up to 25% of the cells infected after 24 hours, while the other three (MGH-U3, MGH-U4 and VMCUB-3) were resistant to BCG infection, with less than 2% of the cells infected after 24 hours (FIG. 1B).

Confocal microscopy confirmed the findings from flow cytometry: cell lines such as MGH-U3, MGH-U4 and VMCUB-3 had almost no visible intracellular GFP positive bacteria, whereas susceptible cell lines, such as UM-UC-3 and T24, displayed abundant intracellular green fluorescent bacteria (FIG. 1C). The possibility that the “resistant” cell lines (MGH-U3, MGH-U4, and VMCUB-3) were actually infected at the same rate as “sensitive” lines, but underwent rapid apoptosis, leaving a population of uninfected cells after apoptotic death of the infected population was considered. To test this possibility, the cells were stained for exposed phosphatidyl serine by annexin V staining, an early marker of apoptosis, at 4 hours and 24 hours after infection, and the proportion of apoptotic cells was determined by flow cytometry (FIG. 1D). The proportion of apoptotic cells was not higher in the BCG-resistant cell lines compared to the BCG-sensitive cell lines, suggesting that apoptosis did not account for the differences in BCG permissiveness between cell lines.

### BCG Uptake by Bladder Cancer Cells is Inhibited by Cytochalasin D, EIPA and Staurosporine

The data obtained indicates that a subset of bladder cancer cells efficiently take up BCG. This result is surprising insofar as bladder cells are non-phagocytic, and mycobacteria, in contrast to other bacterial pathogens such as Salmonella and Listeria, do not have pathogenic effector functions to invade epithelial cells. Thus, the mechanism of BCG uptake into bladder cancer cells susceptible to BCG infection is unclear. To determine the endocytic pathway mediating uptake of BCG in bladder cancer cells, a panel of small molecule inhibitors, which are commonly used to investigate mechanisms of pathogen internalization (19) were utilized, and their effect on uptake of BCG by BCG-permissive cell lines was assessed. The chemical inhibitors used in this study are summarized in the supplementary table. The actin polymerization inhibitor cytochalasin D was tested first and it was found that it diminished uptake of BCG in all three cell lines by 64% to 89% (FIG. 2). Additionally, the Na+/H+ pump inhibitor ethyl-isopropyl amiloride (EIPA), which has been used as an inhibitor of macropinocytosis (20), inhibited uptake of BCG in all cell lines by 47% to 61%. To test the involvement of protein kinases in BCG uptake, the protein kinase inhibitor staurosporine was tested; staurosporine inhibited uptake of BCG in all cell lines by 25% to 46%. In the cell line J82, but not in T24 or UM-UC-3, BCG uptake was also significantly inhibited by genistein (tyrosine-kinase inhibitor). BCG uptake was not inhibited by the nonmuscle myosin inhibitor blebbistatin, which inhibits cell blebbing, or Gö-6983 (a protein kinase C inhibitor). Taken together these data suggest that the uptake of BCG by bladder cancer cells is dependent on the actin cytoskeleton and on protein kinases. The inhibition by EIPA is suggestive of, albeit not specific for, uptake by macropinocytosis (20).

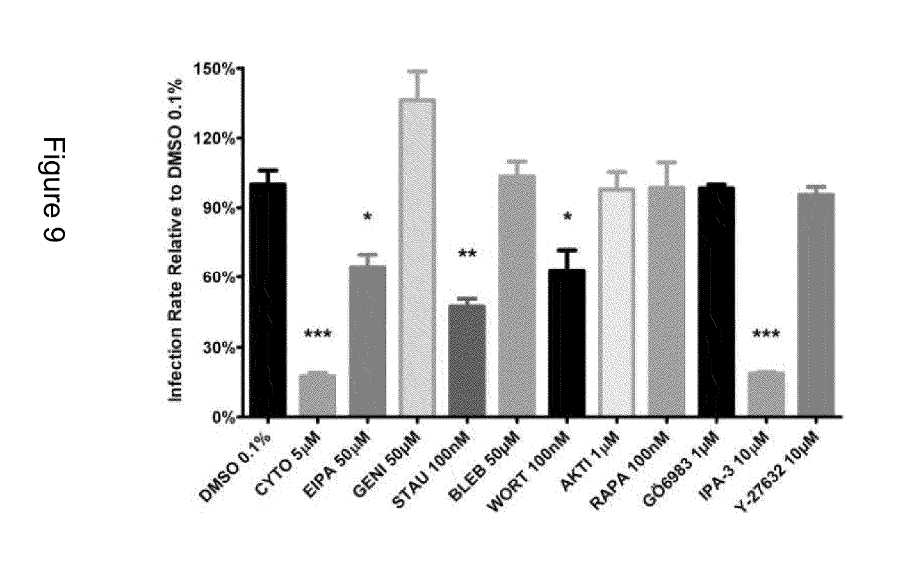

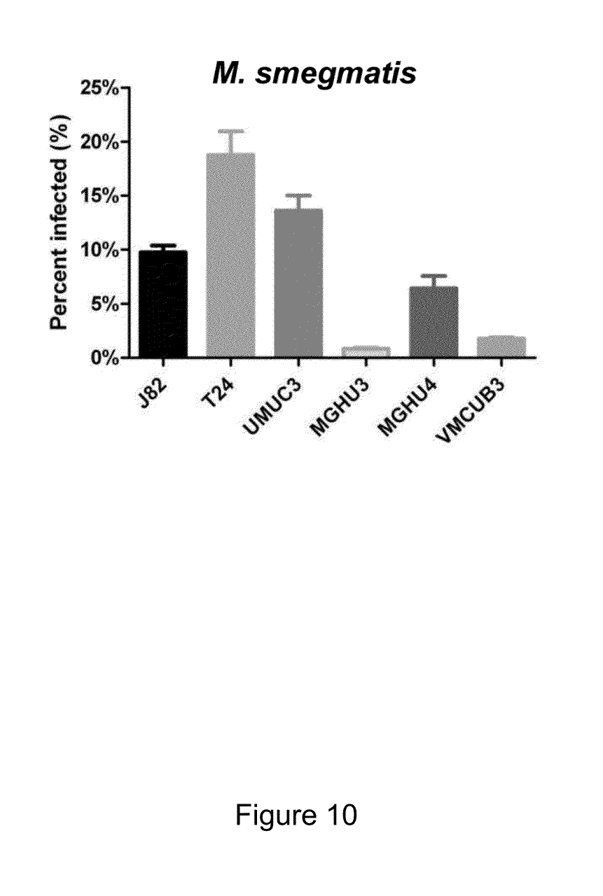

To verify that the action of the inhibitors was not mediated through a direct effect on BCG, the uptake of paraformaldehyde-fixed BCG-GFP by bladder cancer cells in the presence of the same panel of small molecule inhibitors was assessed. The same effects seen with live BCG were also seen with fixed BCG, confirming that the inhibitors were acting through an effect on bladder cancer cells and not through a direct effect on BCG such as compromising bacterial viability (FIG. 9). Of note, these experiments also indicate that the internalization of BCG by bladder cancer cells does not require viable bacteria, seemingly excluding an active pathogen effector function in the uptake process. This conclusion was strengthened by infecting our cell lines with GFP-expressing M. smegmatis. At 4 hours, bladder cancer cell lines infected with M. smegmatis showed the same general pattern as with BCG-BCG-sensitive cell lines were sensitive to, and BCG-resistant cell lines were resistant to M. smegmatis infection (FIG. 10). Thus, the uptake of mycobacteria by bladder cancer cells extends to a nonpathogenic organism.

### BCG Uptake is Dependent on Cdc42, Rac1 and Pak1

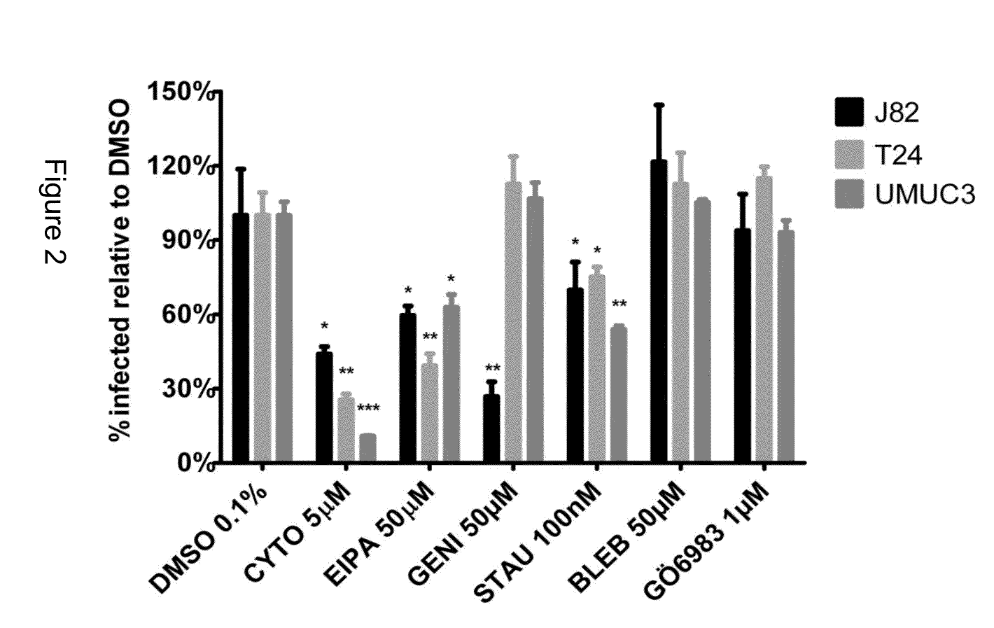

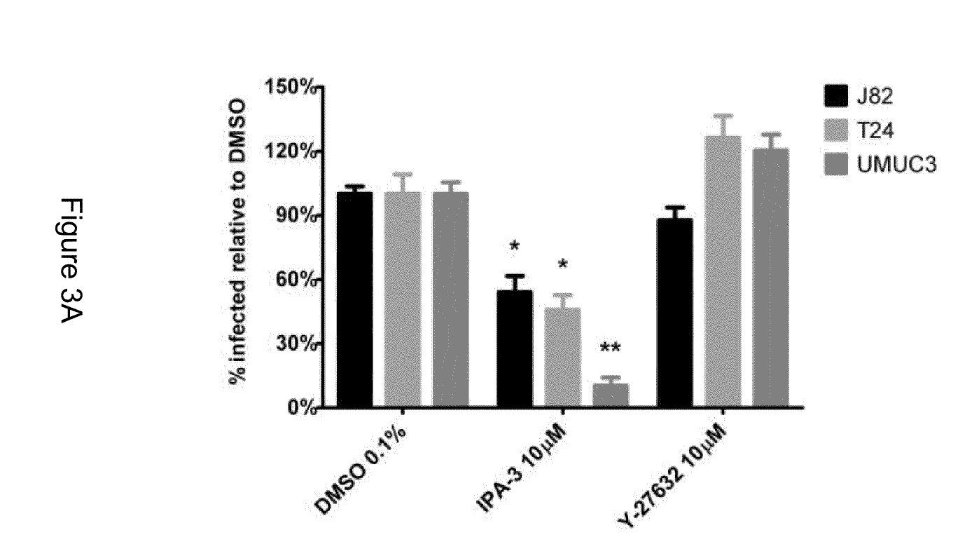

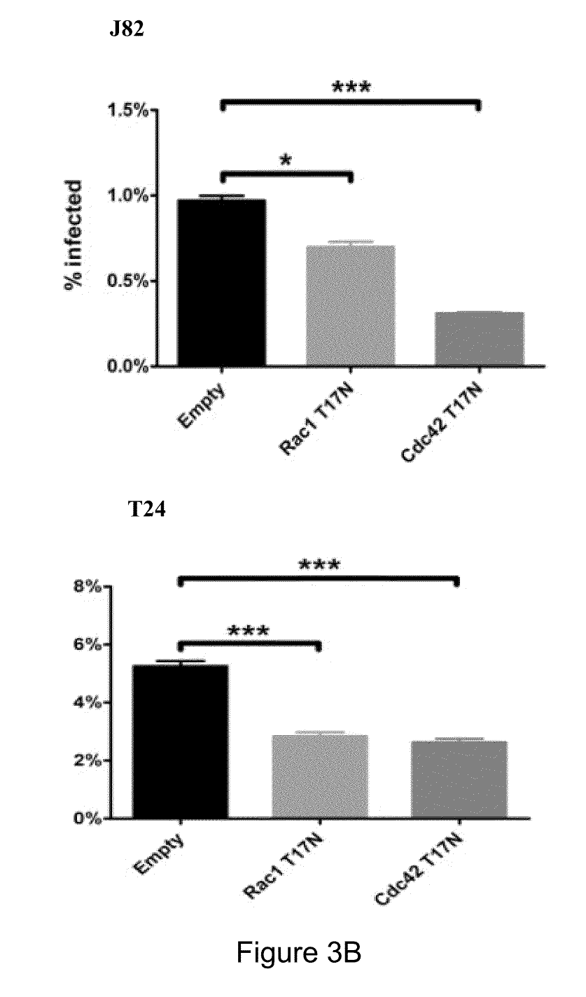

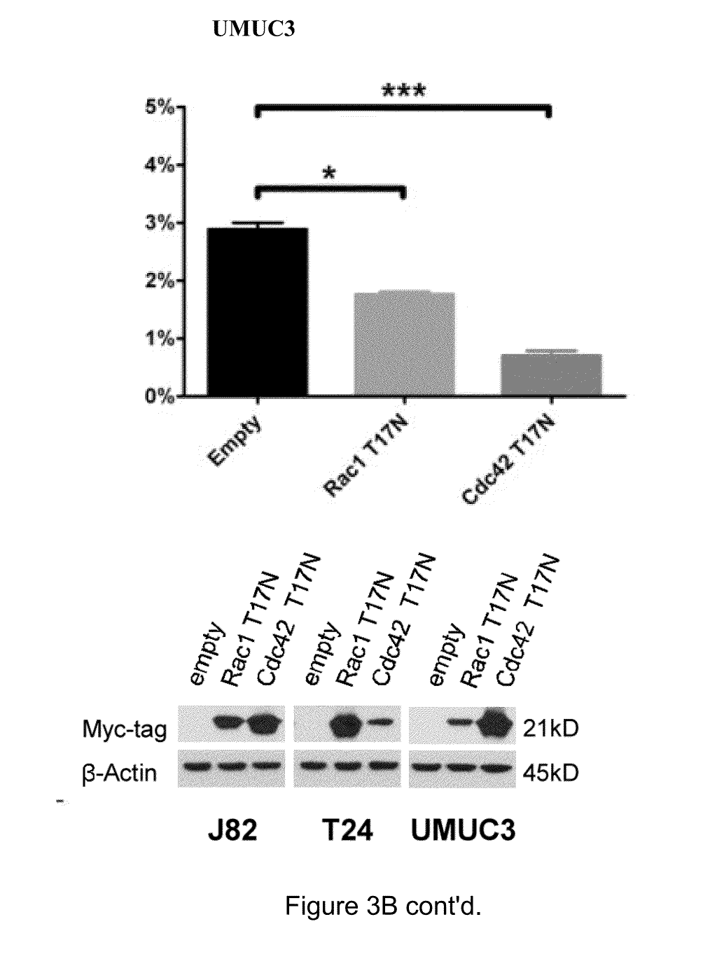

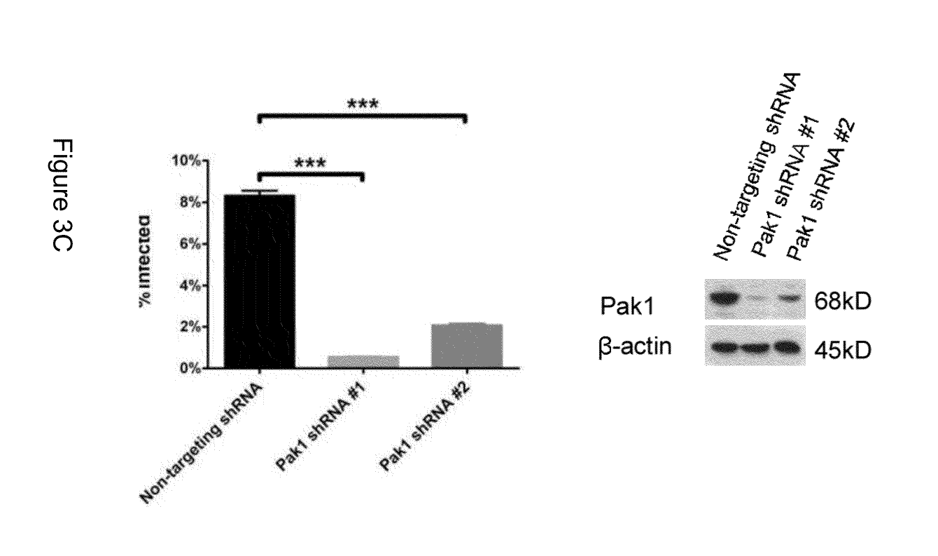

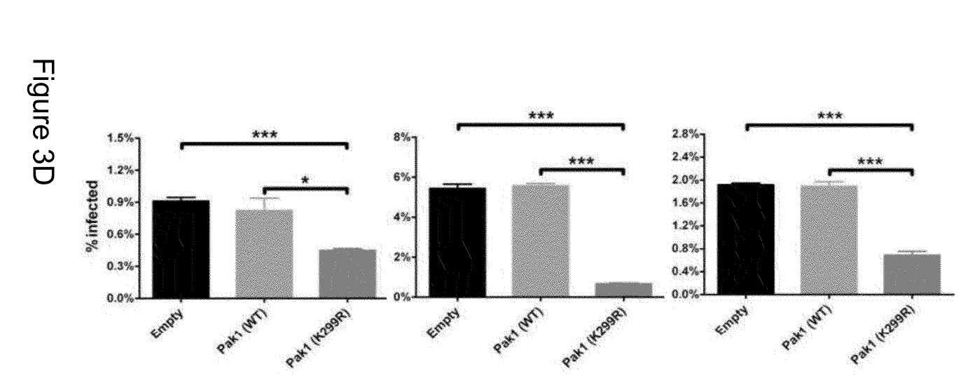

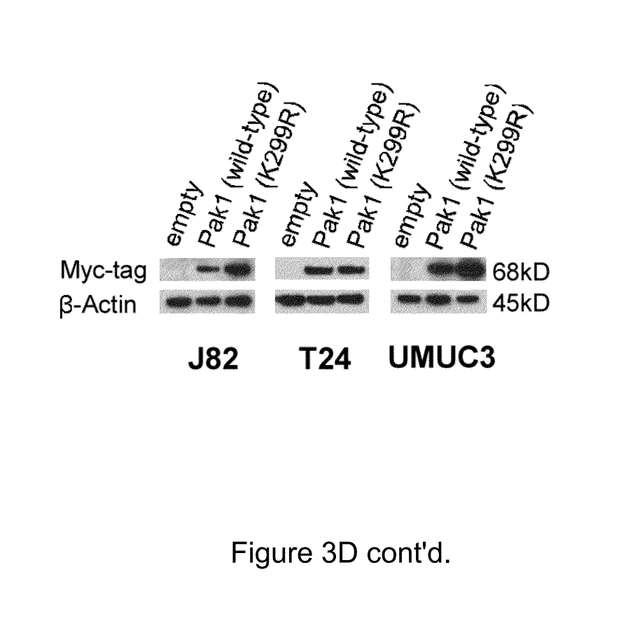

Rho-family GTPases, including Rac1, Cdc42 and RhoA, are involved in actin cytoskeletal organization and in various pathways of endocytosis. Rac1 and Cdc42 control lamellipodia formation and membrane ruffling, and are essential for macropinocytosis and for Fc receptor-mediated phagocytosis (12, 20, 21), as is their downstream effector, p21-activated kinase 1 (Pak1) (22). RhoA, through its downstream effector RhoA Kinase (ROCK), is required for complement receptor-mediated phagocytosis (21). To determine the role of Rho-family GTPases in BCG uptake by bladder cancer cells, we initially used two small molecule inhibitors, Y-27632, an inhibitor of ROCK, and IPA-3, an inhibitor of Pak1. Y-27632 did not have a significant effect on BCG uptake by bladder cancer cells. In contrast, IPA-3 inhibited BCG uptake by 46%-90% (FIG. 3A). As before, we tested the effects of these inhibitors on uptake of fixed BCG, and found that the same effects seen with live BCG occur with fixed BCG, confirming that the inhibitors were acting through their effect on bladder cancer cells (FIG. 9).

To further investigate the role of Rac1 and Cdc42 in BCG uptake, we used dominant negative forms of these two GTPases. We stably transfected BCG-permissive bladder cancer cell lines with Rac1(T17N) and Cdc42(T17N), dominant-negative forms of Rac1 and Cdc42 respectively (13), and measured BCG uptake. We observed that either construct inhibited BCG uptake; Cdc42(T17N) by 50%-75% and Rac1(T17N) by 28%-46% (FIG. 3B), indicating that both contribute to BCG uptake.

The Pak1 protein was depleted by lentiviral delivery of two distinct shRNAs targeting Pak1. Depletion of the Pak1 protein was verified by Western blotting of whole cell lysates with anti-Pak1 antibodies and no effect on Pak1 protein was observed in cells infected with a control scrambled shRNA (FIG. 3C). The effect of Pak1 depletion in the cell line UM-UC-3 was examined. Depletion of Pak1 by either shRNA resulted in striking inhibition of BCG uptake by a factor of 4 to 15 (FIG. 3C), an effect similar to that seen with the Pak1 inhibitor IPA-3. To further confirm this result, BCG-permissive cell lines were stably transfected with Pak1(K299R), a dominant-negative (DN) form of Pak1 (23). Consistent with these results using shRNA, DN-Pak1 decreased BCG uptake by 50%-88% when compared to the same cell line transfected with an empty construct or a wild-type Pak1 construct (FIG. 3D). These data indicate that the BCG entry into permissive bladder cancer cells occurs through a pathway that involves Rac1, Cdc42, and Pak1.

### Uptake of BCG is Independent of Dynamin and Clathrin

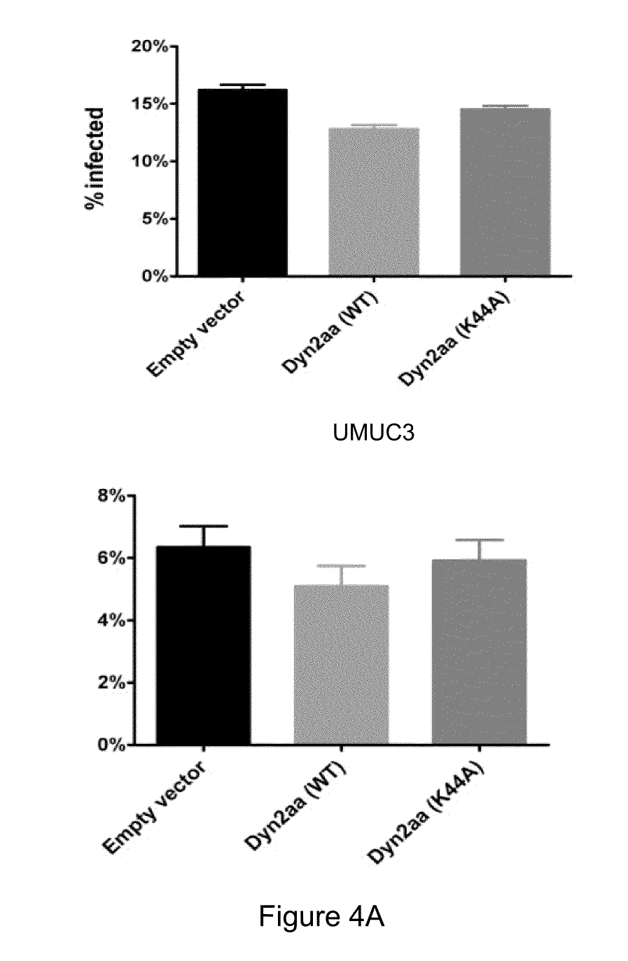

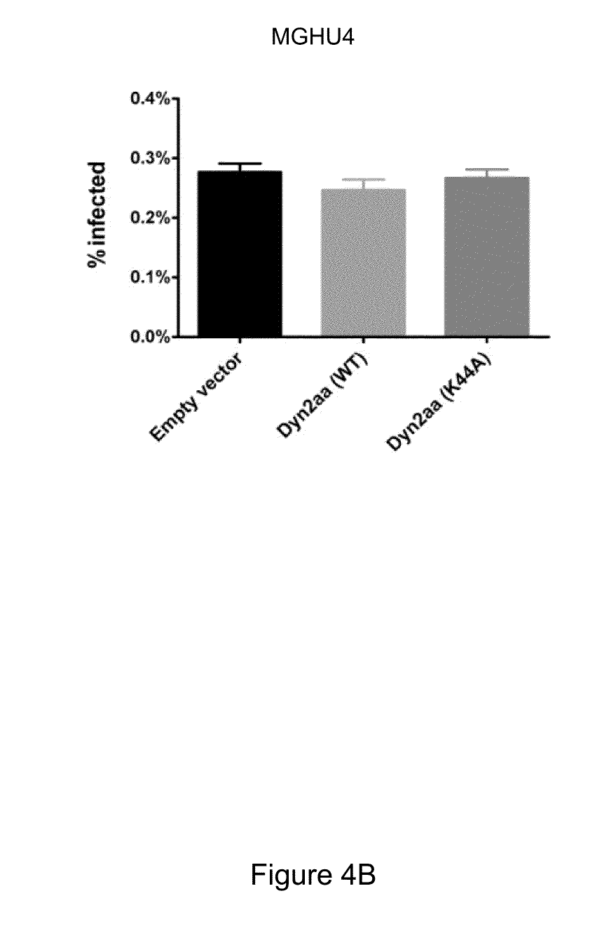

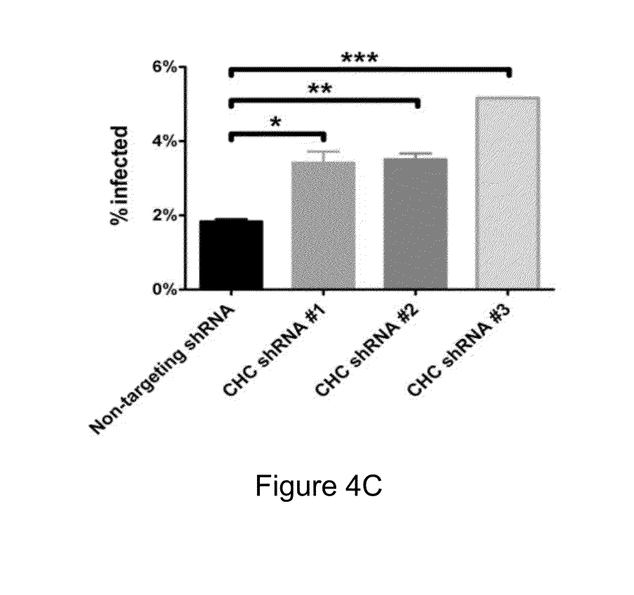

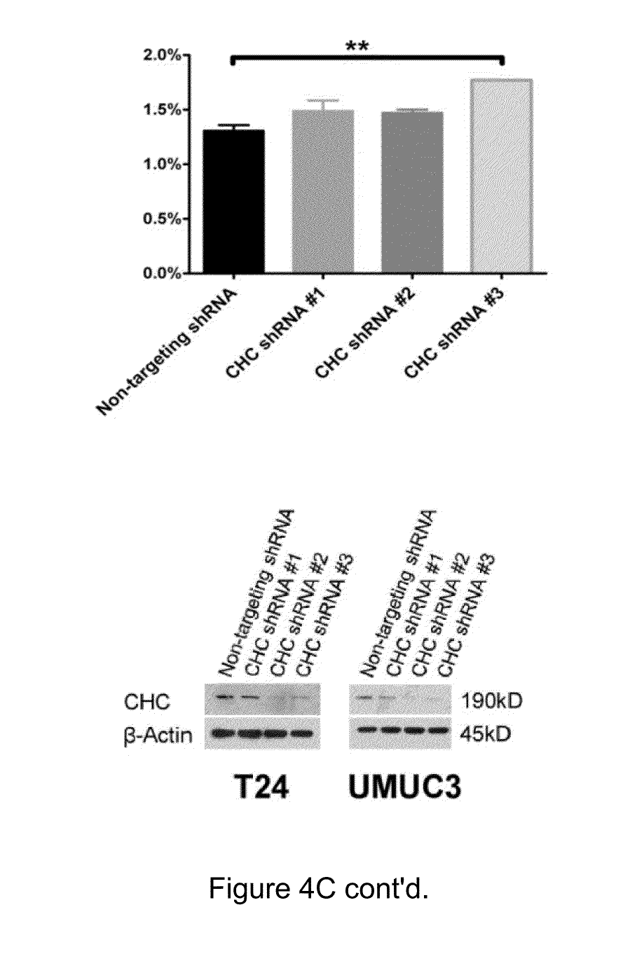

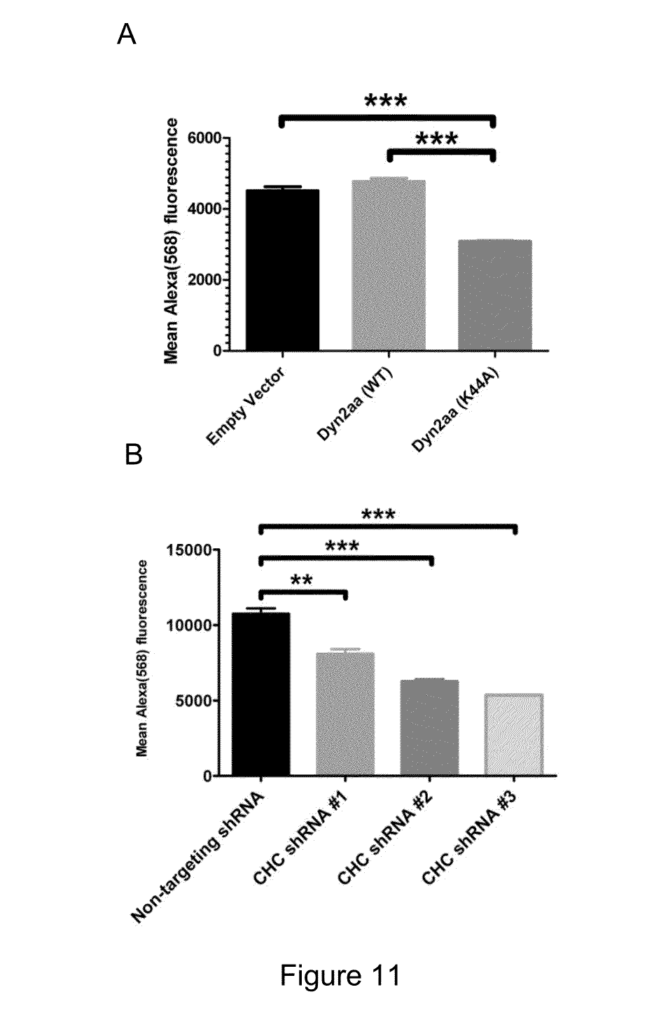

Receptor mediated pathways for the uptake of large particles, such as phagocytosis, or the zippering-type endocytosis used to internalize pathogens such as Listeria, are dependent on the GTPase dynamin (24, 25). To establish whether dynamin is involved in uptake of BCG by bladder cancer cells, we transiently transfected the BCG-sensitive cells lines T24 and UM-UC-3 with wild-type dynamin 2, or the dominant-negative mutant dynamin 2 (K44A) (18). As the C-terminus of dynamin in these constructs is fused to GFP, we used BCG-mCherry in place of BCG-GFP for these experiments. As shown in FIG. 4A, transfection with either construct did not significantly alter uptake of BCG. Similarly, neither construct had an appreciable impact on BCG-mCherry uptake by the BCG-resistant cell line, MGH-U4 (FIG. 4B). Conversely, transfection with dynamin 2 (K44A), but not wild-type dynamin 2, significantly inhibited uptake of transferrin, a process that is known to be dynamin-dependent (26) (FIG. 11A). Clathrin has also been shown to be essential for the “zippering”-type endocytosis of pathogens such as Listeria (25). We determined the role of clathrin in BCG uptake by knocking down clathrin heavy chain in T24 and UM-UC-3 using lentiviral shRNA, and assessing uptake of BCG-GFP. Despite effective knockdown of clathrin heavy chain, as evidenced by Western blotting, no reduction in BCG uptake was observed; in some cases, increased uptake was noted (FIG. 4C). Uptake of transferrin, a known clathrin-dependent process (27), was significantly inhibited by all the clathrin heavy chain shRNA constructs used (FIG. 11B).

Internalized BCG Co-Localizes with Fluid Phase Fluorescent Dextran

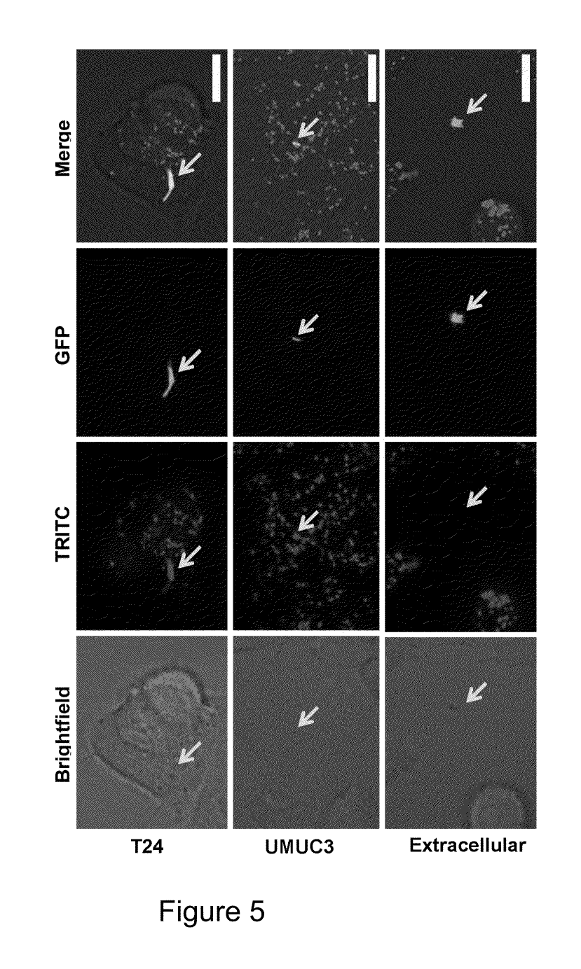

The molecular requirements for BCG uptake, namely the involvement of Rac1/Cdc42/Pak1, the inhibition of uptake by EIPA, and the lack of dependence on dynamin or clathrin, suggest that the pathway of uptake is macropinocytosis. We sought further confirmation of this model by assessing whether fluid phase markers co-localize with BCG. Generally, particles internalized by macropinocytosis are taken up together with extracellular fluid. Conversely, in receptor-mediated uptake pathways, such as phagocytosis or “zippering”, the particles are tightly surrounded by membrane, excluding extracellular fluid (28). To determine whether extracellular fluid is being internalized with BCG, we infected the cell lines T24 and UM-UC-3 with BCG-GFP in the presence of red-fluorescent dextran (MW 10,000) in the medium, and imaged the cells 4 hours later using confocal microscopy. The images clearly indicate that these cells have abundant pinocytotic vesicles marked by fluorescent dextran (FIG. 5). In addition, we observed red fluorescent dextran present in the same vesicle as BCG-GFP (FIG. 5). We excluded the possibility that dextran was attaching to BCG before uptake by observing that fluorescent dextran did not co-localize with BCG that was extracellular (FIG. 5).

### The PI3K-PTEN Pathway Determines BCG Uptake by Bladder Cancer Cells

The data presented above indicate that the mechanism of entry of BCG into some bladder cancer cells is via macropinocytosis. However, some bladder cancer cells are resistant to BCG uptake, suggesting that they do not have an activated macropinocytosis pathway. It was hypothesized that the pattern of mutations present in the BCG-resistant and BCG-sensitive cell lines may determine their permissiveness for BCG uptake. Of the cell lines used in this study, two BCG-permissive cell lines (J82 and UM-UC-3) are reported to have a deletion of PTEN (29), and two (T24 and UM-UC-3) have activating mutations in Ras (30). We investigated the causal relationship between these mutations and BCG susceptibility, focusing first on the PTEN/PI3K/Akt pathway.

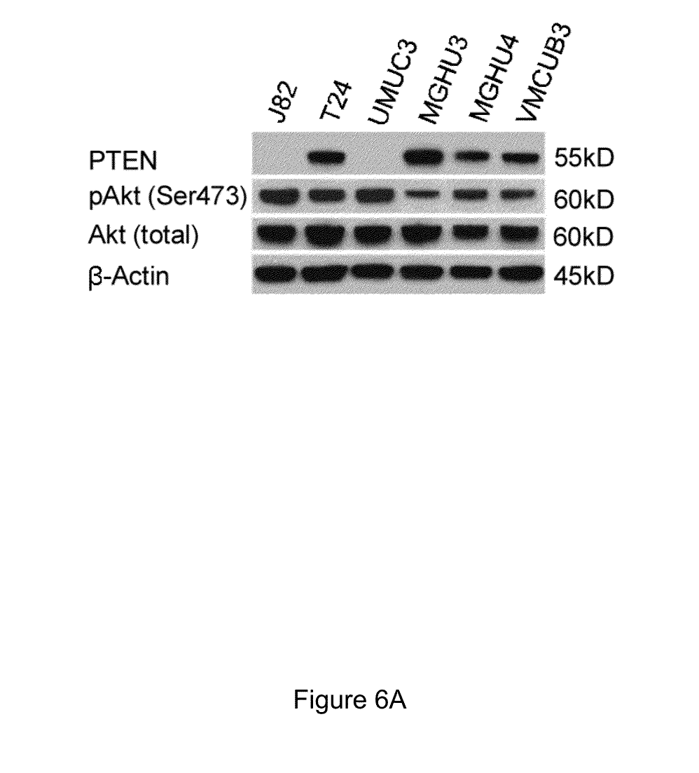

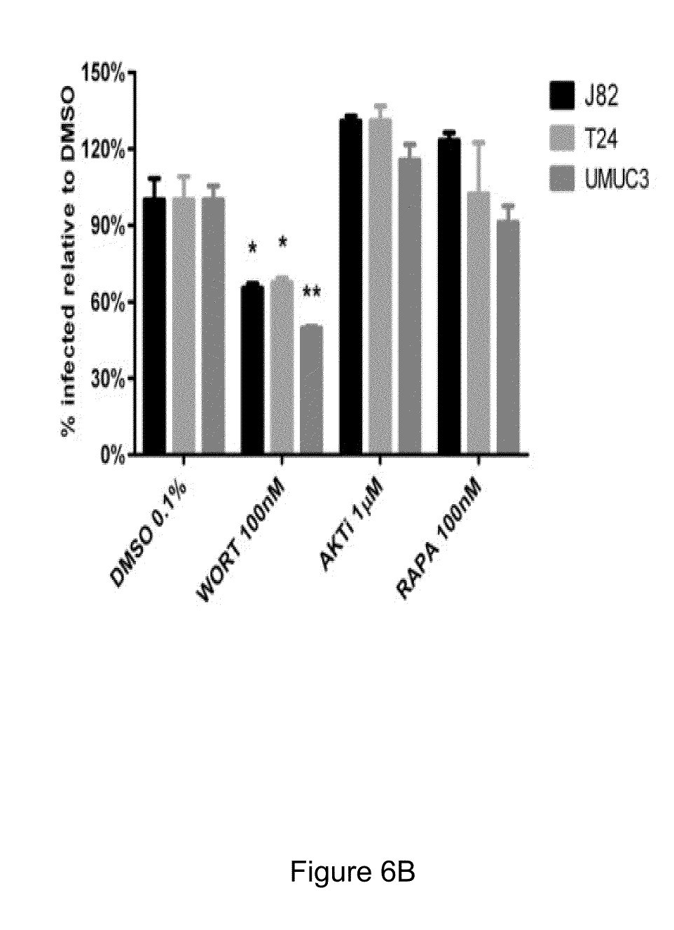

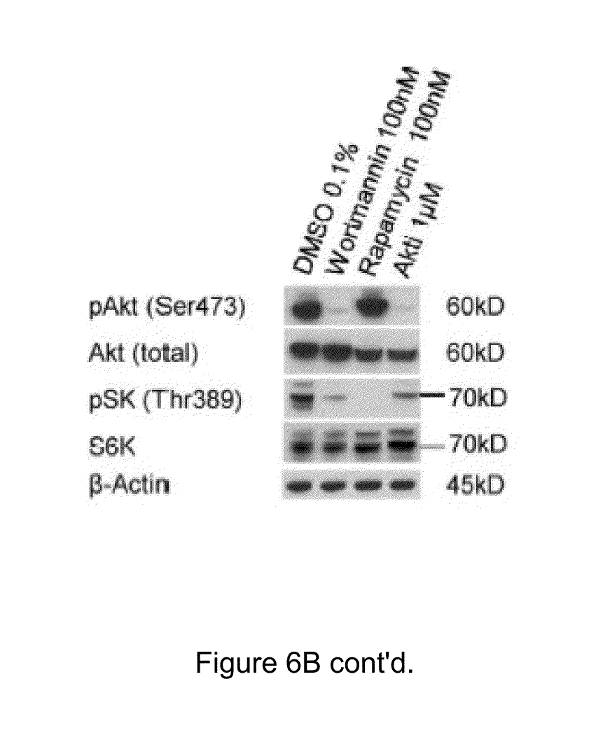

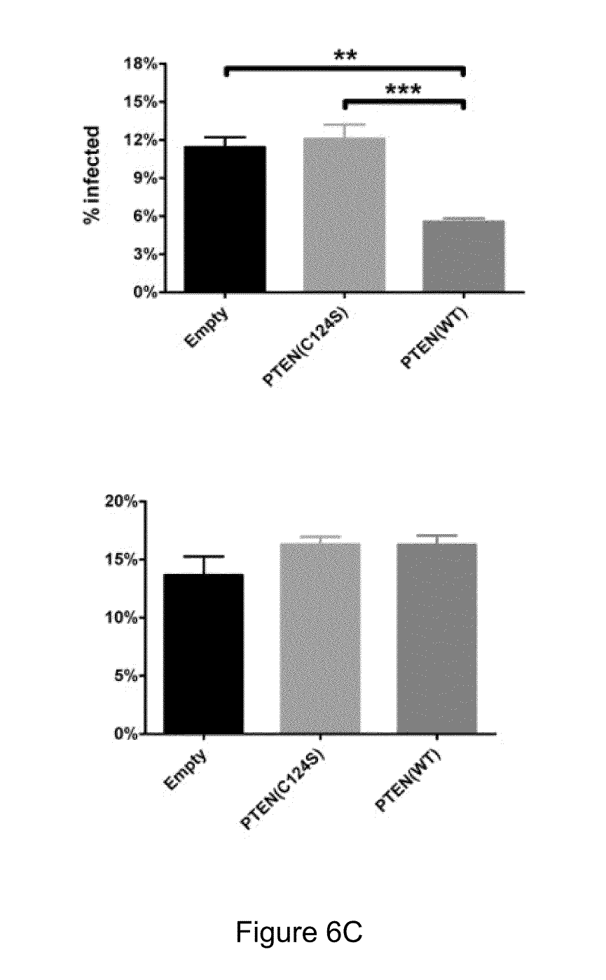

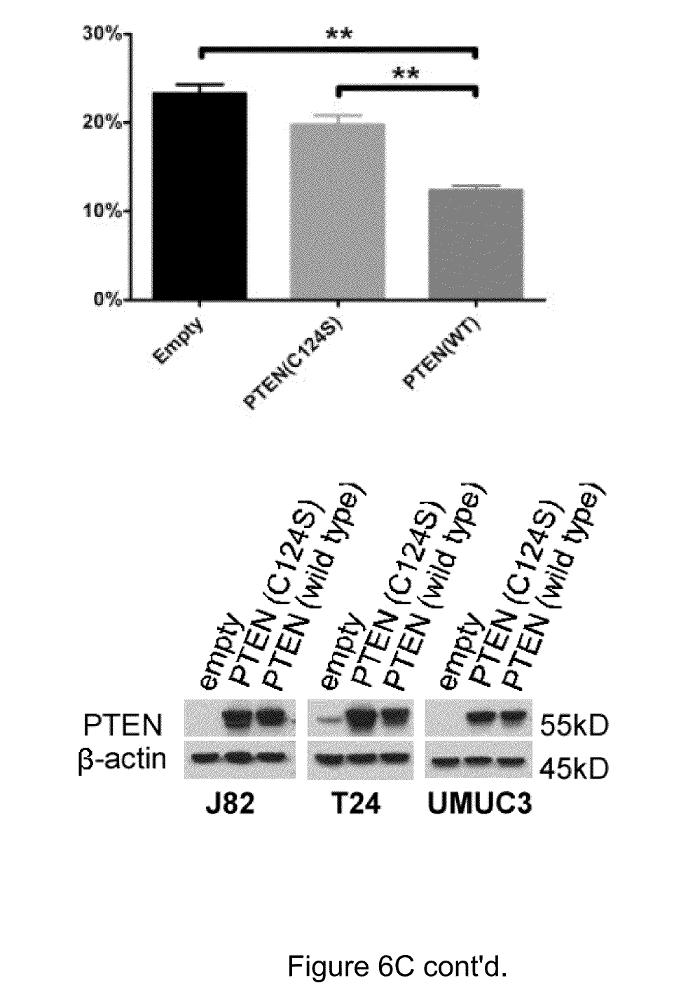

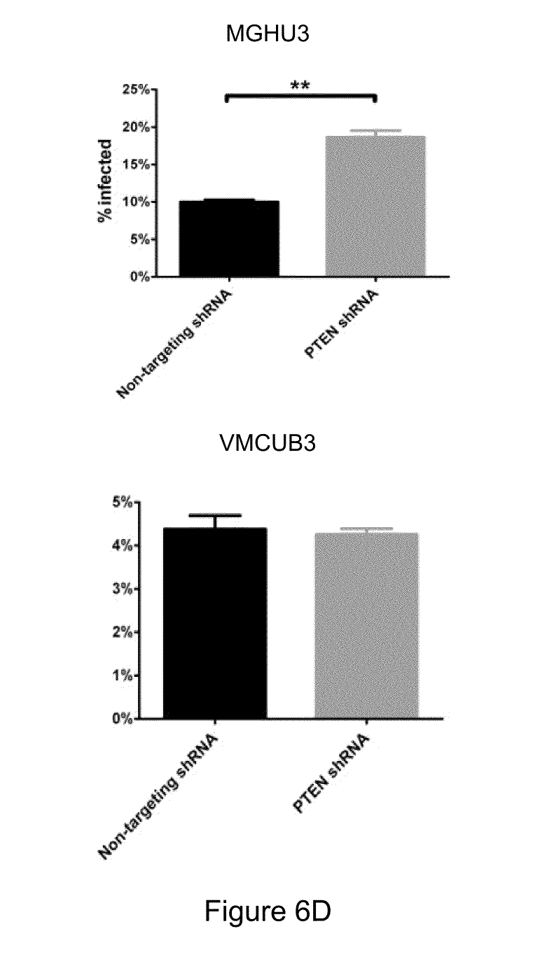

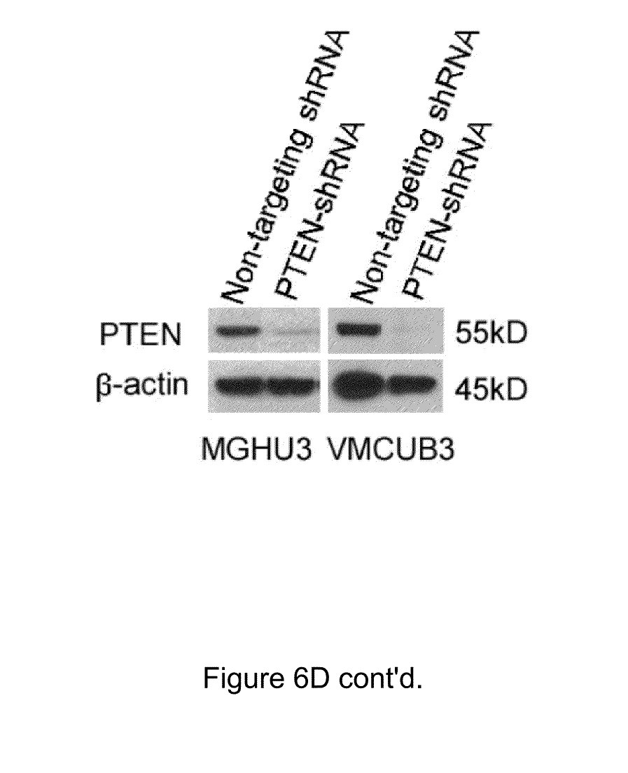

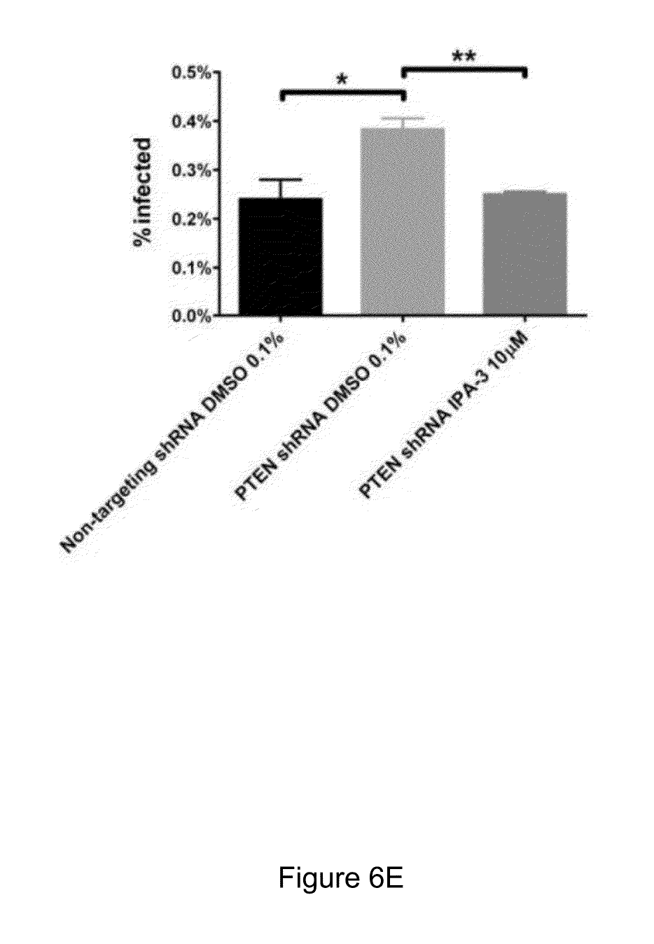

We began investigating the role of the PTEN/PI3K/Akt pathway in BCG uptake by bladder cancer cells by assessing the expression of individual components of the pathway in each of our cell lines using Western blotting (FIG. 6A). As reported, J82 and UM-UC-3, the two cell lines with PTEN deletion, showed no detectable PTEN protein. We proceeded to examine the effects of chemical inhibitors of the pathway on BCG uptake (FIG. 6B). Exposure to wortmannin, an inhibitor of PI3K, resulted in a 33%-50% decrease in BCG uptake in cell lines tested. In contrast, inhibitors of Akt, a major downstream target of PI3K, or mTOR, one of the main targets of Akt, did not decrease BCG uptake by the cells, despite clear evidence of inhibition of their downstream phosphorylation targets (FIG. 6B). We deduced that these inhibitors were not acting through a direct effect on BCG by showing the same effects on uptake of fixed BCG (FIG. 9).

To study the role of PTEN in BCG uptake, we transfected cDNAs encoding PTEN (wild-type) or PTEN (C124S), a PTEN protein deficient for lipid phosphatase activity, into the three BCG sensitive cell lines (FIG. 6C). Intriguingly, induction of wild-type PTEN resulted in approximately 50% reduction in BCG uptake in the cell lines J82 and UM-UC-3, both of which contain a homozygous deletion of PTEN, but not in T24, which expresses PTEN protein, but has been reported to have a missense mutation of PTEN (asparagine to isoleucine at position 48) (29). To determine whether loss of PTEN function could stimulate BCG uptake in a resistant cell line, we knocked down PTEN in the cell lines MGH-U3 and VMCUB-3. Knockdown of PTEN in MGH-U3 resulted in an approximately 2-fold increase in uptake of BCG compared to a non-targeting shRNA control, but the same phenomenon was not seen in VMCUB-3, despite substantial reduction in protein expression as evidenced by Western blotting (FIG. 6D).

The increase in BCG uptake observed with PTEN knockdown could be via macropinocytosis or via another pathway. To confirm that PTEN knockdown was activating the same pathway of BCG uptake observed in permissive cell lines, we tested whether the increase in BCG uptake following PTEN knockdown in the cell line MGH-U4 could be abrogated by inhibition of Pak1. We found that IPA-3 completely abrogated the increase in BCG uptake in the setting of PTEN knockdown (FIG. 6E), indicating that hyperactivation of the PI3K pathway in a resistant cell line activates BCG uptake through the same pathway as in susceptible cell lines with loss of PTEN function. Overall, these data demonstrate that the PTEN-PI3K signaling pathway modulates BCG uptake by bladder cancer cells. Activation of the PI3K pathway activates macropinocytotic uptake of BCG, but this effect is independent of the downstream kinases Akt and mTOR.

### Activated Ras Increases BCG Uptake

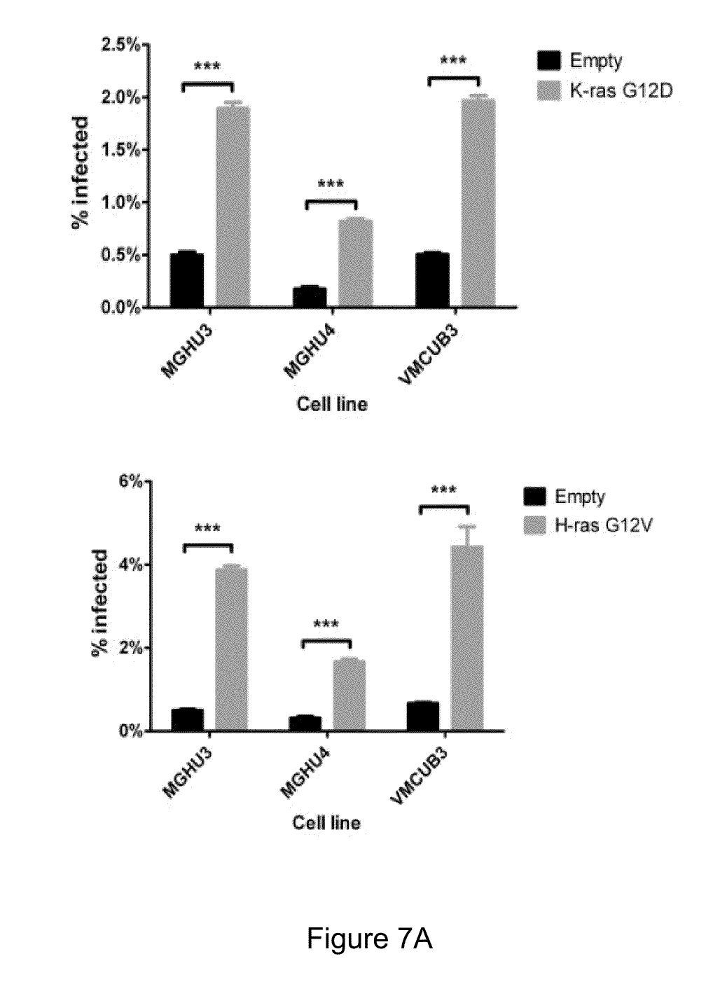

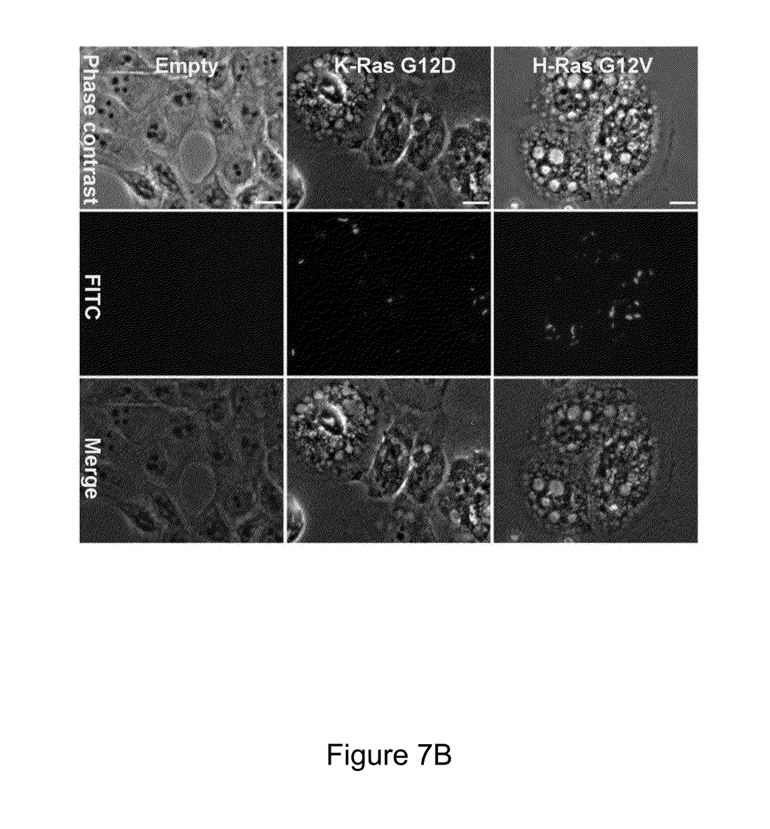

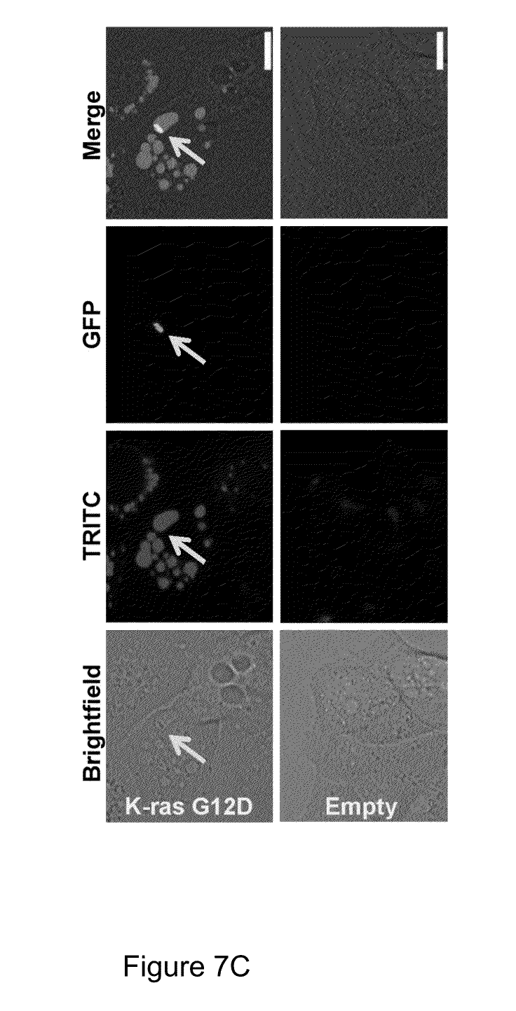

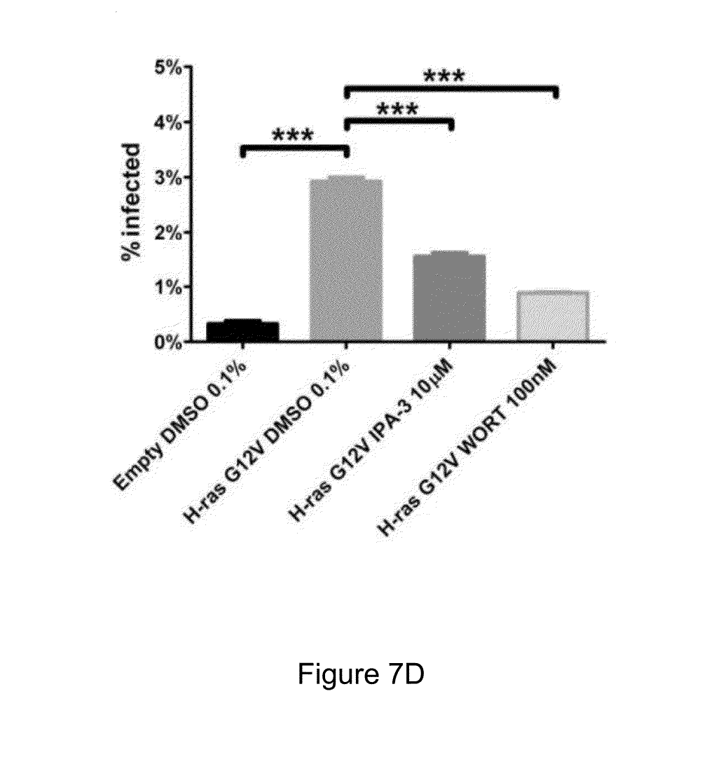

Given that 2 of 3 susceptible cell lines have an activating mutation in Ras, the role of Ras in uptake of BCG by bladder cancer cells was investigated. BCG-resistant cell lines were stably transfected with cDNAs encoding K-Ras G12D and H-Ras G12V, constitutively activated forms of K-Ras and H-Ras, respectively. Both activated forms of Ras caused a dramatic increase in BCG uptake, up to 7-fold higher compared to control cells (FIG. 7A). Fluorescence microscopy confirmed increased BCG uptake by Ras-transformed cell lines, and revealed striking morphologic changes in these cells, including numerous cytoplasmic vacuoles visible by phase-contrast microscopy (FIG. 7B). To determine whether BCG is contained within these vacuoles, Ras-transformed cells were infected with BCG-GFP in the presence of red-fluorescent dextran (MW 10,000) in the culture medium, and the cells were imaged by confocal microscopy. Cells with K-ras G12D, but not control cells, had numerous dextran-containing macropinosomes. BCG could clearly be seen within these dextran-containing vacuoles (FIG. 7C), indicating uptake by macropinocytosis. To confirm that constitutively activated Ras was stimulating the same pathway of BCG uptake observed in permissive cell lines, we tested whether the activation of BCG uptake by Kras G12D could be abrogated by inhibition of PI3K or of Pak1. It was found that IPA-3 and wortmannin each inhibited increased BCG uptake in a cell line with activated Ras, suggesting that the action of Ras occurs upstream of PI3K and Pak1 (FIG. 7D).

### Bladder Cancer Cell Lines

The bladder cancer cell lines J82, T24, UM-UC-3, MGHU-3, MGH-U4, and VMCUB-3 were a kind gift from Dr. Dan Theodorescu. Cells were grown in Eagle minimal essential medium (MEM) supplemented with 10% fetal bovine serum (FBS), 1 mM sodium pyruvate, 2 mM L-glutamine and 1% non-essential amino acids, and with 100 U/ml penicillin, and 100 μg/ml streptomycin (except where noted). Cells were cultured as monolayers at 37° C. in a humidified atmosphere of 5% CO₂ in air. All cells were confirmed to be mycoplasma free by a commercially available mycoplasma detection assay.

Isolation of Exfoliated Bladder Cancer Cells from Urine

To assess the optimal conditions for isolation of bladder cancer cells from patients with bladder cancer, urine specimens were obtained from 10 patients with bladder cancer who underwent cystoscopy at the MSKCC Surgical Day Hospital (SDH). For each patient, urine was obtained prior to the procedure, and an additional sample was obtained through barbotage of the bladder during cystoscopy. The samples were washed and centrifuged, and the cells were divided into three wells of a 24-well plate and were resuspended in three types of cell culture media: (a) MEM with 20% fetal bovine serum (FBS); (b) DMEM with 20% FBS; (c) KSFM with 25 μg/ml bovine pituitary extract+5 ng/ml epidermal growth factor. Cells were incubated for a time sufficient for cells to attach to the plate, for example, from about 12 to about 72 hours.

Growth of cells occurred in 5 of 10 samples allowed to attach overnight. However, only in 1 of these 5 samples were the cells confirmed by an expert cytopathologist to be consistent with malignant cells; the remainder were morphologically consistent with normal urothelial cells. The type of media used did not have a significant impact on yield of cells. The yield of cells from voided urine was higher than it was for barbotage. In general, the number of attached cells was low and was considered insufficient for evaluation of BCG uptake by flow-cytometry. For these samples, microscopy was chosen as the method to determine uptake of BCG.

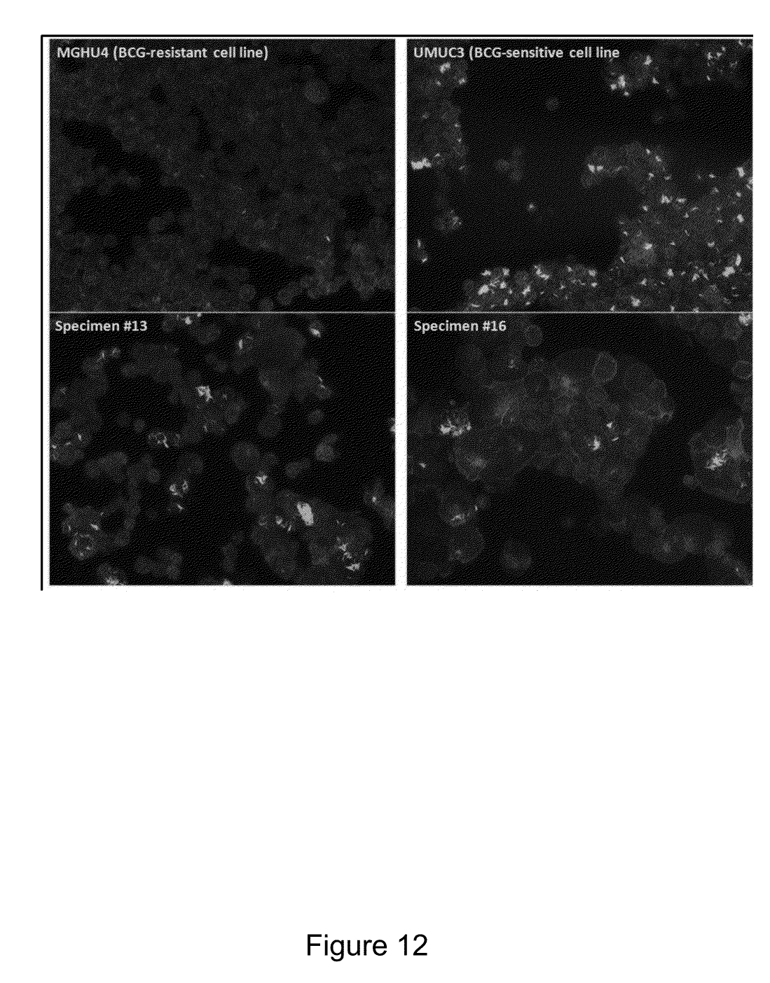

Evaluation of BCG uptake by bladder cancer cells: Urine was obtained from 23 additional patients with bladder cancer. Using the growth conditions as described above, we were able to demonstrate growth of cells in 14 of 23 samples. Once again, the number of cells obtained was low (<1,000 cells per patient). 7 of the 14 samples contained cells that were morphologically consistent with malignant urothelial cells based on an evaluation by an expert cytopathologist. All samples with cell growth were infected with GFP-expressing BCG for 24 hours. As controls, we concurrently infected bladder cancer cell lines, one that is sensitive to BCG uptake and one that is resistant. An example of two patient specimens is shown in FIG. 12, which shows representative images of BCG uptake in bladder cancer cells. Patient specimens #13 and #16, and bladder cancer cell line controls MGHU4 (BCG-resistant) and UMUC3 (BCG-sensitive) were infected with GFP-expressing BCG for 24 hours. No fluorescence is seen in BCG-resistant cell line, MGHU4, while fluorescence due to BCG uptake is evident in BCG-sensitive cell line, UMUC3 and both patient specimens.

BCG and Mycobacterium smegmatis

GFP-expressing BCG (BCG-GFP) was created by transforming Mycobacterium bovis Calmette Guerin Pasteur strain with pYUB921 (an episomal plasmid encoding GFP and conferring kanamycin resistance). mCherry-expressing BCG (BCG-mCherry) was created by transforming BCG Pasteur with pMSG432 (an episomal plasmid encoding mCherry and conferring hygromycin resistance). BCG strains were grown at 37° C. in Middlebrook 7H9 media supplemented with 10% albumin/dextrose/saline (ADS), 0.5% glycerol and 0.05% Tween 80, and in the presence of 20 μg/ml kanamycin (BCG-GFP) or 50 μg/ml of hygromycin (BCG-mCherry). To create titered stocks for infection, the BCG strains were grown to mid-log phase (OD600 0.4-0.6), washed twice in phosphate-buffered saline (PBS) with 0.05% Tween 80, resuspended in PBS with 25% glycerol, and stored at −80° C. To measure final bacterial titer, an aliquot was thawed, and serial dilutions were plated on 7H10 plates in the presence of 20 μg/ml kanamycin (BCG-GFP) or 50 μg/ml of hygromycin (BCG-mCherry), and the bacterial titer determined by counting kanamycin/hygromycin resistant colonies after 3 weeks of incubation.

GFP-expressing M. smegmatis was created by transforming M. smegmatis with pYUB921. M. smegmatis was grown in LB media supplemented with 0.5% glycerol, 0.5% dextrose, and 0.05% Tween 80, in the presence of 20 μg/ml kanamycin. Tittered stocks for infection were created as described for BCG.

### BCG Infection

Bladder cancer cells were plated a day prior to infection in antibiotic-free media so as to reach 50%-80% confluence on the day of infection. Cells were washed with serum-free antibiotic-free media, and media was replaced with serum-free antibiotic-free media for one hour prior to infection. BCG was thawed and diluted in serum-free antibiotic-free media to achieve an MOI (multiplicity of infection) of 10:1. Plates were incubated at 37° C. for the specified time period and then washed three times with PBS, and three times with antibiotic-containing media (with 1′)/0 penicillin-streptomycin). Cells were washed once again with PBS, detached using trypsin, and resuspended in PBS for analysis by flow cytometry.

### Flow Cytometry

Cell suspensions derived from BCG infection were analyzed on an LSR II flow cytometer (BD Biosciences), using the FACS DiVa software (BD Biosciences) according to manufacturer's instructions. Data analysis was performed with the FlowJo software package (Tree Star). GFP was detected on the FITC channel using a 488 nm laser. mCherry was detected on the PE-Texas Red channel using a 532 nm laser. As the cell lines had a high degree of auto-fluorescence, an empty channel (Pacific Blue) was used to optimize gating of GFP-positive or mCherry positive events. The gating strategy is described in FIG. 8.

### Pharmacologic Inhibitors

The pharmacological inhibitors used in this study are detailed in the supplementary table. The cells were pre-treated with the inhibitors in serum free media at the specified concentrations for one hour prior to infection with BCG, and kept in the media for the duration of infection. In all experiments utilizing chemical inhibitors, the highest concentration of DMSO (0.1%) was used as vehicle control.

### Plasmids and Transfections

PLK0.1-PTEN and PLK0.1-SC were a gift from Dr. Xuejun Jiang. PLK0.1-Pak1 and PLK0.1-clathrin heavy chain shRNA constructs were purchased from the Memorial Sloan Kettering High-Throughput Screening core facility. The scrambled shRNA lentivirus PLK0.1-SC was used as control for shRNA knockdown.

The sequences for expression of shRNA for PTEN, Pak1 and Clathrin Heavy Chain were as follows:

PTEN shRNA:
SEQ ID NO: 1
5′-CCGGCCACAGCTAGAACTTATCAAACTCGAGTTTGATAAGTTCT
AGCTGT-3′
Pak1 shRNA #1:
SEQ ID NO: 2
5′-CCGGGCATTCGAACCAGGTCATTCACTCGAGTGAATGACCTGGT
TCGAATGCTTTTTTG-3′
Pak1 shRNA #2:
SEQ ID NO: 3
5′-CCGGGAGCTGCTACAGCATCAATTCCTCGAGGAATTGATGCTGT
AGCAGCTCTTTTTTG-3′
Clathrin heavy chain shRNA #1:
SEQ ID NO: 4
5′-CCGGCGTGTTCTTGTAACCTTTATTCTCGAGAATAAAGGTTACA
AGAACACGTTTTT-3′
Clathrin heavy chain shRNA #2:
SEQ ID NO: 5
5′-CCGGGCCCAAATGTTAGTTCAAGATCTCGAGATCTTGAACTAAC
ATTTGGGCTTTTT-3′
Clathrin heavy chain shRNA #3:
SEQ ID NO: 6
5′-CCGGCCTGTGTAGATGGGAAAGAATCTCGAGATTCTTTCCCATC
TACACAGGTTTTT-3′

The lentiviral constructs pQCXIP-Rac1 (T17N) (12) and pQCXIP-Cdc42 (T17N) (13) were a kind gift from Dr. Alan Hall. pcDNA3.1-PTEN (wild type) and PTEN (C124S) (14) were provided by Dr. Xuejun Jiang. PTEN cDNA was amplified from these constructs and cloned into pQCXIP-IRES-puro using the BamHI and EcoRI restriction sites. The polyadenylation site AATAAA in both inserts was mutated synonymously to AACAAG. PCMV6-Pak1 (WT), Pak1 (T423E) and Pak1 (K299R) (15) were a generous gift from Dr. Jonathan Chernoff. The constructs were cut with the restriction enzymes BamHI and EcoRI, and the Pak1 cDNA fragment was cloned into pQCXIP-IRES-puro using the BamHI and EcoRI restriction sites. RCAS-K-ras (G12D) (16) and PWZL-H-ras (G12V) (17) were kindly given by Dr. Eric Holland. K-ras and H-ras cDNA was amplified from these constructs and cloned into pQCXIP-IRES-puro using the BamHI and EcoRI restriction sites. All amplified inserts were sequenced prior to cloning to confirm that no mutations arose during amplification. The empty lentivirus pQCXIP-IRES-puro was used as control for overexpression constructs.

Lentivirus for shRNA knockdown of PTEN, Pak1 or clathrin heavy chain was made by co-transfecting the respective plasmids with Mission Lentiviral Packaging Mix (Sigma) into 293T cells in 10 cm² plates, using lipofectamine 2000 (Invitrogen) as per the manufacturer's instructions. Lentivirus for overexpression of PTEN, Pak1 Cdc42, Rac1, K-ras and H-ras was made by co-transfecting the respective constructs with the packaging plasmids VSV-G and pCPG into 293T cells in 10 cm² plates, using lipofectamine 2000. A day prior to infection with lentivirus, bladder cancer cell lines were plated at 1×10⁵ per well in 6-well plates and allowed to attach overnight. On day of infection media was replaced with supernatant from 293T plates, and polybrene 8 μg/ml (Sigma) was added. Plates were spun at 1100 g for 30 minutes. The media was replaced with fresh antibiotic-free MEM, and the plates were allowed to incubate overnight. The following day cells containing the lentiviral insert were selected using 1.5 μg/ml puromycin (Invitrogen) for 4 days. Cells that had not been infected with lentivirus were used as control for selection.

The dynamin constructs pEGFP-dynamin 2aa (WT) and pEGFP-dynamin 2aa (K44A) (18) were a kind gift from Dr. Mark McNiven. Cells were transiently transfected with the dynamin constructs in 6-well plates, using X-treme Gene HP DNA transfection reagent (Roche) as per the manufacturer's instructions. Infection with BCG was carried out 24 hours after transfection. As these constructs express GFP, BCG-mCherry was used in these experiments.

### Antibodies

Antibodies against pAkt (Ser473, D9E, #4060), Akt (C67E7, #4691), S6K (#9202), p-S6K (Thr389 #9205), Pak1 (#2602), β-actin (8H10D10, #3700), clathrin heavy chain (D3C6, #4796), and Myc-Tag (9B11, #2276) were purchased from Cell Signaling Technology. PTEN antibody (clone 6H2.1) was purchased from Cascade BioScience.

### Microscopy

For microscopy of fixed samples, cells were plated on glass coverslips in 6-well plates and allowed to attach overnight. The following day the cells were washed with serum-free antibiotic-free media, and media was replaced with serum-free antibiotic-free media for one hour prior to infection. BCG was thawed and diluted in serum-free antibiotic-free media to achieve a MOI of 10:1. Plates were incubated at 37° C. for the specified time period, and washed three times with PBS and three times with antibiotic-containing media. Nuclei were stained using Hoechst (Invitrogen) for 10 minutes. Cells were then fixed with 4% PFA at room temperature for 10 minutes, permeabilized with 0.1% Triton X-100 in PBS for 5 minutes, and stained with Texas-Red Phalloidin (Invitrogen). Slides were mounted on microscopy slides with Mowiol mounting medium. Confocal images were obtained with a Leica Inverted confocal SP2 microscope, using Leica acquisition software. A 20× objective (numerical aperture 0.7) or a 63× objective (numerical aperture 1.2) were used. Phase contrast microscopy was conducted using a Zeiss AxioVert 200M microscope with a Coolsnap ES camera, controlled by Metamorph acquisition software version 7.7.4 (Molecular Devices). A 40× objective (numerical aperture 0.6) was used. For live imaging using fluorescent dextran, cells were plated in glass-bottom 35 mm dishes (MatTek) and allowed to attach overnight. The following day the cells were infected with BCG at an MOI of 10:1 as described above. Alexa Fluor 568-conjugated dextran MW 10,000 (Invitrogen) at a concentration of 0.1 mg/ml was added to the media immediately following addition of BCG. The cells were incubated with BCG and fluorescent dextran at 37° C. for the specified time period, and washed three times with PBS and three times with antibiotic-containing media. Live microscopy was performed on a Zeiss Axiovert 200M microscope, with a Yokogawa spinning disk (CSU-22) unit, and an incubation chamber set to 37° C. with 5% CO2 in air. Images were acquired with an Andor iXon+ camera controlled by Metamorph acquisition software version 7.7.4 (Molecular Devices). A 63× oil objective (numerical aperture 1.4) was used.

All microscopes were available through the Memorial Sloan Kettering Molecular Cytology Core Facility. All microscopy images were adjusted for contrast using Volocity software (Perkin Elmer).

### Apoptosis and Cell Death Assay

Bladder cancer cells were infected with BCG-GFP for 4 hours or 24 hours. Cells were then washed once with PBS, detached with trypsin, spun at 1,250 rpm for 5 minutes, and resuspended in PBS. In order to evaluate apoptosis, cells were stained using Pacific Blue Annexin V (Invitrogen) per the manufacturer's instructions. The proportion of apoptotic cells (positive for annexin V fluorescence) was determined by flow-cytometry. Unstained cells were used as controls.

### Transferrin Uptake

Cells were washed with serum-free media, and media was replaced with serum-free media for one hour prior to addition of transferrin. Media was replaced with serum-free media containing 25 μg/ml Alexa-568-conjugated transferrin (Invitrogen) for 15 minutes at 37° C. Internalization was stopped by chilling the cells on ice and washing three times with ice-cold PBS. Cells were then washed with 0.1 M glycine, 0.1 M sodium chloride, PH 3.0 to remove any transferrin that was not internalized. Cells were detached using trypsin, resuspended in PBS, and analyzed by flow-cytometry. Internalized transferrin was detected by the PE-Texas Red channel using a 532 nm laser.

When validated in clinical settings, these findings have implications for the treatment of patients with bladder cancer. Based upon the results, Ras and PTEN aberrations may represent predictive biomarkers of BCG efficacy. Prospective genetic profiling of TUR specimens for mutations within these key oncogenic signaling pathways would allow clinicians to restrict BCG therapy to those patients most likely to respond. Furthermore, as novel therapies targeting oncogenic pathways are being developed for the treatment of bladder cancer, such as inhibitors of PI3K (49), receptor tyrosine kinase inhibitors (50) and RNA-interference mediated silencing of Cdc42 (38), care should be taken to consider possible effects of these treatment on BCG uptake and efficacy. Finally, BCG therapy could possibly be improved by local administration of activators of these pathways, thereby potentially rendering BCG-resistant cells sensitive. The findings presented here will catalyze a direct examination of the role of specific macropinocytosis activating mutations in clinical response to BCG.

In conclusion, it is shown that BCG uptake by bladder cancer cells is determined by some of the same pathways that lead to oncogenesis. Knowledge of the mechanism underlying responsiveness to BCG therapy helps tailor the treatment to individual patients based on their tumor genotype, and leads to the development of more effective treatment options for bladder cancer.

### REFERENCES

1. Jemal A, Siegel R, Xu J, Ward E. Cancer statistics, 2010. C A Cancer J Clin. 2010; 60:277-300.
2. Kirkali Z, Chan T, Manoharan M, Algaba F, Busch C, Cheng L, et al. Bladder cancer: epidemiology, staging and grading, and diagnosis. Urology. 2005; 66:4-34.
3. Gontero P, Bohle A, Malmstrom P U, O'Donnell M A, Oderda M, Sylvester R, et al. The role of bacillus Calmette-Guerin in the treatment of non-muscle-invasive bladder cancer. Eur Urol. 2010; 57:410-29.
4. Bohle A, Brandau S. Immune mechanisms in bacillus Calmette-Guerin immunotherapy for superficial bladder cancer. J Urol. 2003; 170:964-9.
5. Pook S H, Rahmat J N, Esuvaranathan K, Mahendran R. Internalization of Mycobacterium bovis, Bacillus Calmette Guerin, by bladder cancer cells is cytotoxic. Oncol Rep. 2007; 18:1315-20.
6. Becich M J, Carroll S, Ratliff T L. Internalization of bacille Calmette-Guerin by bladder tumor cells. J Urol. 1991; 145:1316-24.
7. Kavoussi L R, Brown E J, Ritchey J K, Ratliff T L. Fibronectin-mediated Calmette-Guerin bacillus attachment to murine bladder mucosa. Requirement for the expression of an antitumor response. The Journal of clinical investigation. 1990; 85:62-7.
8. Ikeda N, Toida I, Iwasaki A, Kawai K, Akaza H. Surface antigen expression on bladder tumor cells induced by bacillus Calmette-Guerin (BCG): A role of BCG internalization into tumor cells. International journal of urology: official journal of the Japanese Urological Association. 2002; 9:29-35.
9. Cossart P, Sansonetti P J. Bacterial invasion: the paradigms of enteroinvasive pathogens. Science. 2004; 304:242-8.
10. Veiga E, Guttman J A, Bonazzi M, Boucrot E, Toledo-Arana A, Lin A E, et al. Invasive and adherent bacterial pathogens co-Opt host clathrin for infection. Cell host & microbe. 2007; 2:340-51.
11. Ginocchio C, Pace J, Galan J E. Identification and molecular characterization of a Salmonella typhimurium gene involved in triggering the internalization of salmonellae into cultured epithelial cells. Proc Natl Acad Sci USA. 1992; 89:5976-80.
12. Nobes C D, Hall A. Rho, rac, and cdc42 GTPases regulate the assembly of multimolecular focal complexes associated with actin stress fibers, lamellipodia, and filopodia. Cell. 1995; 81:53-62.
13. Nobes C D, Hall A. Rho GTPases control polarity, protrusion, and adhesion during cell movement. The Journal of cell biology. 1999; 144:1235-44.
14. Maccario H, Perera N M, Gray A, Downes C P, Leslie N R. Ubiquitination of PTEN (phosphatase and tensin homolog) inhibits phosphatase activity and is enhanced by membrane targeting and hyperosmotic stress. The Journal of biological chemistry. 2010; 285:12620-8.
15. Sells M A, Knaus U G, Bagrodia S, Ambrose D M, Bokoch G M, Chernoff J. Human p21-activated kinase (Pak1) regulates actin organization in mammalian cells. Current biology: CB. 1997; 7:202-10.
16. Orsulic S, Li Y, Soslow R A, Vitale-Cross L A, Gutkind J S, Varmus H E. Induction of ovarian cancer by defined multiple genetic changes in a mouse model system. Cancer cell. 2002; 1:53-62.
17. Serrano M, Lin A W, McCurrach M E, Beach D, Lowe S W. Oncogenic ras provokes premature cell senescence associated with accumulation of p53 and p16INK4a. Cell. 1997; 88:593-602.
18. Ochoa G C, Slepnev V I, Neff L, Ringstad N, Takei K, Daniell L, et al. A functional link between dynamin and the actin cytoskeleton at podosomes. The Journal of cell biology. 2000; 150:377-89.
19. Mercer J, Helenius A. Vaccinia virus uses macropinocytosis and apoptotic mimicry to enter host cells. Science. 2008; 320:531-5.
20. Koivusalo M, Welch C, Hayashi H, Scott C C, Kim M, Alexander T, et al. Amiloride inhibits macropinocytosis by lowering submembranous pH and preventing Rac1 and Cdc42 signaling. The Journal of cell biology. 2010; 188:547-63.
21. Swanson J A. Shaping cups into phagosomes and macropinosomes. Nature reviews Molecular cell biology. 2008; 9:639-49.
22. Dharmawardhane S, Schurmann A, Sells M A, Chernoff J, Schmid S L, Bokoch G M. Regulation of macropinocytosis by p21-activated kinase-1. Molecular biology of the cell. 2000; 11:3341-52.
23. Zhang S, Han J, Sells M A, Chernoff J, Knaus U G, Ulevitch R J, et al. Rho family GTPases regulate p38 mitogen-activated protein kinase through the downstream mediator Pak1. The Journal of biological chemistry. 1995; 270:23934-6.
24. Veiga E, Cossart P. Listeria hijacks the clathrin-dependent endocytic machinery to invade mammalian cells. Nature cell biology. 2005; 7:894-900.
25. Gold E S, Underhill D M, Morrissette N S, Guo J, McNiven M A, Aderem A. Dynamin 2 is required for phagocytosis in macrophages. The Journal of experimental medicine. 1999; 190:1849-56.
26. van der Bliek A M, Redelmeier T E, Damke H, Tisdale E J, Meyerowitz E M, Schmid S L. Mutations in human dynamin block an intermediate stage in coated vesicle formation. The Journal of cell biology. 1993; 122:553-63.
27. Hanover J A, Willingham M C, Pastan I. Kinetics of transit of transferrin and epidermal growth factor through clathrin-coated membranes. Cell. 1984; 39:283-93.
28. Silverstein S C, Steinman R M, Cohn Z A. Endocytosis. Annual review of biochemistry. 1977; 46:669-722.
29. Wang D S, Rieger-Christ K, Latini J M, Moinzadeh A, Stoffel J, Pezza J A, et al. Molecular analysis of PTEN and MXI1 in primary bladder carcinoma. International journal of cancer Journal international du cancer. 2000; 88:620-5.
30. Jebar A H, Hurst C D, Tomlinson D C, Johnston C, Taylor C F, Knowles M A. FGFR3 and Ras gene mutations are mutually exclusive genetic events in urothelial cell carcinoma. Oncogene. 2005; 24:5218-25.
31. Kodaki T, Woscholski R, Hallberg B, Rodriguez-Viciana P, Downward J, Parker P J. The activation of phosphatidylinositol 3-kinase by Ras. Current biology: C B. 1994; 4:798-806.
32. Lambert J M, Lambert Q T, Reuther G W, Malliri A, Siderovski D P, Sondek J, et al. Tiam1 mediates Ras activation of Rac by a PI(3)K-independent mechanism. Nature cell biology. 2002; 4:621-5.
33. Missy K, Van Poucke V, Raynal P, Viala C, Mauco G, Plantavid M, et al. Lipid products of phosphoinositide 3-kinase interact with Rac1 GTPase and stimulate GDP dissociation. The Journal of biological chemistry. 1998; 273:30279-86.
34. Zheng Y, Bagrodia S, Cerione R A. Activation of phosphoinositide 3-kinase activity by Cdc42Hs binding to p85. The Journal of biological chemistry. 1994; 269:18727-30.
35. Platt F M, Hurst C D, Taylor C F, Gregory W M, Harnden P, Knowles M A. Spectrum of phosphatidylinositol 3-kinase pathway gene alterations in bladder cancer. Clinical cancer research: an official journal of the American Association for Cancer Research. 2009; 15:6008-17.
36. Boulalas I, Zaravinos A, Karyotis I, Delakas D, Spandidos D A. Activation of RAS family genes in urothelial carcinoma. J Urol. 2009; 181:2312-9.
37. Volanis D, Zaravinos A, Kadiyska T, Delakas D, Zoumpourlis V, Spandidos D A. Expression profile of Rho kinases in urinary bladder cancer. Journal of BUON: official journal of the Balkan Union of Oncology. 2011; 16:511-21.
38. Wu F, Chen Y, Li Y, Ju J, Wang Z, Yan D. RNA-interference-mediated Cdc42 silencing down-regulates phosphorylation of STAT3 and suppresses growth in human bladder-cancer cells. Biotechnology and applied biochemistry. 2008; 49:121-8.
39. Zhang Y H, Xie D, Luo J H, Chen W, Chen L W, Xu Q C, et al. [The clinico-pathological significance of protein expression of PAK1 in bladder transitional cell carcinoma]. Zhonghua yi xue za zhi. 2007; 87:2710-3.
40. Ito M, Nishiyama H, Kawanishi H, Matsui S, Guilford P, Reeve A, et al. P21-activated kinase 1: a new molecular marker for intravesical recurrence after transurethral resection of bladder cancer. J Urol. 2007; 178:1073-9.
41. Ernst J D. Macrophage receptors for Mycobacterium tuberculosis. Infection and immunity. 1998; 66:1277-81.
42. Martinez J J, Hultgren S J. Requirement of Rho-family GTPases in the invasion of Type 1-piliated uropathogenic Escherichia coli. Cellular microbiology. 2002; 4:19-28.
43. Visvikis O, Boyer L, Torrino S, Doye A, Lemonnier M, Lores P, et al. Escherichia coli producing CNF1 toxin hijacks Tollip to trigger Rac1-dependent cell invasion. Traffic. 2011; 12:579-90.
44. Wang Z, Humphrey C, Frilot N, Wang G, Nie Z, Moniri N H, et al. Dynamin2- and endothelial nitric oxide synthase-regulated invasion of bladder epithelial cells by uropathogenic Escherichia coli. The Journal of cell biology. 2011; 192:101-10.
45. Eto D S, Gordon H B, Dhakal B K, Jones T A, Mulvey M A. Clathrin, AP-2, and the NPXY-binding subset of alternate endocytic adaptors facilitate FimH-mediated bacterial invasion of host cells. Cellular microbiology. 2008; 10:2553-67.
46. Ratliff T L, McCarthy R, Telle W B, Brown E J. Purification of a mycobacterial adhesin for fibronectin. Infection and immunity. 1993; 61:1889-94.
47. Amstutz B, Gastaldelli M, Kalin S, Imelli N, Boucke K, Wandeler E, et al. Subversion of CtBP1-controlled macropinocytosis by human adenovirus serotype 3. The EMBO journal. 2008; 27:956-69.
48. Nanbo A, Imai M, Watanabe S, Noda T, Takahashi K, Neumann G, et al. Ebolavirus is internalized into host cells via macropinocytosis in a viral glycoprotein-dependent manner. PLoS pathogens. 2010; 6:e1001121.
49. Wu D, Tao J, Xu B, Qing W, Li P, Lu Q, et al. Phosphatidylinositol 3-kinase inhibitor LY294002 suppresses proliferation and sensitizes doxorubicin chemotherapy in bladder cancer cells. Urologia internationalis. 2011; 86:346-54.
50. Dreicer R, Li H, Stein M, DiPaola R, Eleff M, Roth B J, et al. Phase 2 trial of sorafenib in patients with advanced urothelial cancer: a trial of the Eastern Cooperative Oncology Group. Cancer. 2009; 115:4090-5.

1. A method for determining the responsiveness of a bladder cancer patient to treatment with bacillus Calmette Guerin (BCG), the method comprising:
(a) contacting an isolated bladder cancer cell or cells from the patient with BCG containing a detectable label for a period of time sufficient for said BCG to be internalized by said cell(s);
(b) determining the amount of BCG uptake by said isolated bladder cancer cell(s) or cells from the patient;
(c) comparing the amount of BCG uptake by said isolated bladder cancer cell or cells from the patients with
(i) a reference amount of BCG uptake by normal bladder cells; and/or
(ii) a reference amount of BCG uptake by known BCG-permissive cells; and
(d) determining that the patient will be responsive to therapy with BCG when the amount of labeled BCG taken up by said isolated bladder cancer cell or cells from the patient is greater than the amount taken up by normal bladder cells and/or equal to or greater than the amount of uptake in known BCG-permissive cells.

2. A method for selecting treatment options for a patient with bladder cancer, the method comprising:
(a) contacting an isolated bladder cancer cell or cells from the patient with BCG containing a detectable label for a period of time sufficient for said BCG to be internalized by said cell(s);
(b) determining the amount of BCG uptake by said isolated bladder cancer cell(s) or cells from the patient;
(c) comparing the amount of BCG uptake by said isolated bladder cancer cell or cells from the patient with:
(i) a reference amount of BCG uptake by normal bladder cells, and/or
(ii) a reference amount of BCG uptake by known BCG-permissive cells;
wherein treatment with BCG is indicated when BCG uptake by said isolated bladder cancer cell or cells from the patient is greater than BCG uptake by normal bladder cells or equal to or greater than uptake in known permissive cells.

3. The method of claim 1, wherein said detectable BCG comprises a detectable fluorescent marker.

4. The method of claim 1, wherein said detectable BCG expresses a detectable fluorescent protein marker.

5. The method of claim 4, wherein said detectable marker is selected from green fluorescent protein and mCherry.

6. The method of claim 1, wherein the amount of BCG uptake in said cell is determined by flow cytometry or confocal microscopy.

7. The method of claim 1, wherein said bladder cancer is non-muscle invasive urothelial carcinoma (NMIUC).

8. The method of claim 1, wherein said known BCG-permissive cell is UM-UC-3 or T24 cells.

9. A method for determining the responsiveness of a patient with bladder cancer to treatment with bacillus Calmette Guerin (BCG), the method comprising:
determining the presence of any one of the following in a bladder cancer cell isolated from the patient:
(a) decreased expression or deletion of PTEN;
(b) an activating mutation of Ras,
(c) overexpression of Pak1; or
(d) elevated expression of Cdc42 compared to the level of Cdc42 expression in normal urothelial cells,
wherein the presence of any one of (a), (b), (c), or (d) or a combination thereof indicates that the patient will be responsive to treatment with BCG.

10. The method of claim 9, wherein the activating mutation of Ras is a K-ras, H-Ras or N-ras mutation.

11. The method of claim 10, wherein the mutation is selected from K-Ras G12D or H-Ras G12V.

12. The method of claim 9, wherein said bladder cancer is non-muscle invasive urothelial carcinoma (NMIUC).

13. A kit comprising:
(a) BCG that comprises a detectable label; and
(b) a known BCG responsive cell.

14. The method of claim 11, wherein said detectable label is a fluorescent label.

15. The method of claim 11, wherein said BCG expresses a fluorescent protein.

16. The method of claim 12, wherein said fluorescent protein is green fluorescent protein or mCherry.

17. The method of claim 11, wherein said known BCG responsive cell is UM-UC-3 or T24.

18. A method for identifying an agent that enhances BCG uptake by bladder cancer cells, the method comprising:
(a) contacting a known resistant bladder cancer cell with an agent;
(b) contacting said known resistant bladder cancer cell with BCG containing a detectable label for a period of time normally sufficient for said BCG to be internalized by permissive cell(s);
(b) determining the amount of BCG uptake by said known resistant bladder cancer cell;
(c) comparing the amount of BCG uptake by said known resistant bladder cancer cell with
(i) a reference amount of BCG uptake by normal bladder cells;
(ii) a reference amount of BCG uptake by known BCG-permissive cells; and/or
(iii) a reference amount of BCG uptake by the known BCG-resistant cell prior to exposure with the agent;
(d) determining that the agent tested enhances BCG uptake by bladder cancer cells when the amount of BCG uptake in said cell is greater than the amount of BCG uptake by normal cells or resistant cells not exposed to agent or equal to or greater than the reference amount of BCG uptake by known BCG-permissive cells.

19. The method of claim 18, wherein said agent activates a component of a Ras and/or PI3K pathway.


SEQUENCE LISTING (6 sequences)

SEQ ID NO: 1 - DNA, 50 bp, Artificial Sequence
  synthetic construct
ccggccacagctagaacttatcaaactcgagtttgataagttctagctgt

SEQ ID NO: 2 - DNA, 59 bp, Artificial Sequence
  synthetic construct
ccgggcattcgaaccaggtcattcactcgagtgaatgacctggttcgaatgcttttttg

SEQ ID NO: 3 - DNA, 59 bp, Artificial Sequence
  synthetic construct
ccgggagctgctacagcatcaattcctcgaggaattgatgctgtagcagctcttttttg

SEQ ID NO: 4 - DNA, 57 bp, Artificial Sequence
  Synthetic construct
ccggcgtgttcttgtaacctttattctcgagaataaaggttacaagaacacgttttt

SEQ ID NO: 5 - DNA, 57 bp, Artificial Sequence
  synthetic construct
ccgggcccaaatgttagttcaagatctcgagatcttgaactaacatttgggcttttt

SEQ ID NO: 6 - DNA, 57 bp, Artificial Sequence
  synthetic construct
ccggcctgtgtagatgggaaagaatctcgagattctttcccatctacacaggttttt